(19) United States
(12) Patent Application Publication
(10) Pub. No.: US 2019/0274978 A1
(43) Pub. Date: Sep. 12, 2019

(54) ANTIBACTERIAL COMPOSITIONS

(71) Applicants: JANINE NAOMI COPP, Nelson (NZ); VICTORIA LINK LIMITED, Wellington (NZ)

(72) Inventors: David Francis Ackerley, Wellington (NZ); Janine Naomi Copp, Nelson (NZ)

(21) Appl. No.: 16/302,534

(22) PCT Filed: May 18, 2017
(86) PCT No.: PCT/NZ2017/050065

§ 371 Date: Nov. 16, 2018

(30) Foreign Application Priority Data
May 18, 2016 (NZ) 720296

(51) Int. Cl. A61K 31/166; A61K 31/426; A61P 31/04; A61K 38/12; A61K 9/00

(52) U.S. Cl. CPC A61K 31/166; A61K 31/426; A61K 9/0019; A61K 38/12; A61P 31/04

(57) ABSTRACT

The present invention provides compositions, including pharmaceutical compositions, comprising a salicylamide compound and an agent that increases the permeability of a bacterial cell membrane for use in treating or preventing bacterial infections.

### TECHNICAL FIELD

The present invention relates generally to salicylamide compounds in combination with agents that increase the permeability of a bacterial cell membrane, as well as compositions thereof, effective in the prevention or treatment of bacterial infections.

### BACKGROUND OF THE INVENTION

It is widely expected that the rise of multi-drug resistant bacteria will be the largest health concern facing humans in the 21^{st} century (Boucher et al. (2009) Clinical Infectious Diseases 48(1):1-12; Piddock (2012) Lancet Infect Dis. 12(3):249-53). Clinicians are already regularly faced with cases of antibiotic resistance, with previously simple to treat infections becoming more difficult and in some cases impossible to treat. Nearly all classes of antibiotics were discovered before 1970 and over the last 30 years no new major classes of antibiotics have been developed. Most advances for new antibiotic therapies have recently been within antibiotic classes, through the development of analogues to known antibiotics. However, resistance mechanisms have developed so that now whole classes of antibiotics are ineffective against certain bacteria.

To decrease the rate of antibiotic resistance, greater measures are being taken to limit the spread and incidence of infection, together with education on the proper use of antibiotics and limiting their use in ways that promote the development of infection. However, there is still a need for new antibiotics, in particular antibiotics that are effective against Gram negative bacteria, which represent a significant proportion of infectious disease burden.

Niclosamide (N-(2′-chloro-4′-nitrophenyl)-5-chlorosalicylamide) is a salicylanilide compound. Salicylanilides were identified as useful for killing snails following the screening of 20,000 compounds against the snail Biomphalaria glabrata in the 1950s and structural optimisation (Gonnert (1961) Results of laboratory and field trials with the molluscicide Bayer 73. Sun and Zhang (Sun and Zhang (1999) Tubercle and Lung Disease 79(5): 319-320) investigated antifungal and antihelmintic drugs for activity against Mycobacterium tuberculosis, broadly classified as a Gram-positive bacteria, although it possesses “acid fast” cell wall characteristics of both Gram-positive and Gram negative bacteria. They found niclosamide to be very active against M. tuberculosis, with an MIC of 0.5-1.0 μg/mL. Niclosamide was active against non-replicating M. tuberculosis grown in low oxygen conditions, which currently accounts for the lengthy treatment of M. tuberculosis infections. These authors did observe toxicity against macrophages grown in tissue culture. Salicylanilide analogues of niclosamide have been screened to further investigate their use in M. tuberculosis treatment (Krátký, et al. (2010) European Journal of Medicinal Chemistry 45(12):6106-6113; Krátký, et al. (2012) Tuberculosis 92(5):434-439)

de Carvalho et al. also investigated niclosamide and the structural analogue nitazoxanide for efficacy against M. tuberculosis (de Carvalho et al. (2011) ACS Medicinal Chemistry Letters 2(11):849-854). They showed that niclosamide and nitazoxanide uncoupled the membrane potential of M. tuberculosis, whereas a control, rifampicin, did not.

In a recent screen of a commercially available FDA-approved drug library to identify compounds active in inhibiting the growth of methicillin-resistant Staphylococcus aureus (MRSA), Lau et al. showed that niclosamide was an effective anti-MRSA agent, with a sub-micromolar minimum inhibitory concentration under the assay conditions used (Lau et al. (2015) Antibiotics (Basel) 4(4):424-34).

Niclosamide and the related salicylanilide anthelmintic drug oxyclozanide were also found by Rajamuthiah et al. to be directly toxic to MRSA, as well as another Gram-positive bacteria Enterococcus faecium (Rajamuthiah et al. (2015). PloS One 10(4):e0124595). This work was inspired by the authors' earlier finding that another related salicylanilide compound, closantel, was also active against MRSA (Rajamuthiah et al. (2014) PLoS One 9(2):e89189). However, Rajamuthiah et al. specifically noted that neither niclosamide nor oxyclozanide were active in inhibiting the growth of any of the Gram negative bacteria strains tested (i.e. Klebsiella pneumoniae, Acinetobacter baumannii, Pseudomonas aeruginosa and Enterobacter aerogenes).

The potential of niclosamide as an indirect inhibitor of Gram negative pathogenesis was recently studied by Imperi et al., who screened FDA-approved drugs to identify any inhibitors of the quorum sensing system in Pseudomonas aeruginosa (Imperi et al. (2013) Antimicrobial Agents Chemotherapy 57(2):996-1005). Of the drugs tested, niclosamide exhibited the highest anti-quorum sensing activity. Further analysis determined that niclosamide was able to inhibit the response to the quorum sensing signal rather than the synthesis of the signal molecule. However, the authors did not consider a directly toxic role for niclosamide, and their data was not consistent with niclosamide having any directly toxic effects against P. aeruginosa. In fact, nuclosamide failed to inhibit the growth of P. aeruginosa in the assays reported (e.g. FIG. 2A).

Applicants recently discovered that niclosamide and related salicylamides are surprisingly directly toxic to Gram negative bacteria when administered in combination with an efflux pump inhibitor (e.g.) TolC efflux pump inhibitor (PCT/NZ2015/050192; unpublished). This finding was unknown to researchers in the field of bacteriology and represents a significant advance toward development of combination therapies against Gram negative bacteria.

Combination therapies involving treatments with synergistic mechanisms of action, are one of the most promising strategies to combat the rise in antibiotic resistance (Lee et al. (2016) J Pharm Sci 105:1501-1512). Combination therapies are favored for two predominant characteristics: (1) synergistic effect, where the combined effect of two compounds is greater than the sum of their individual effects, e.g., therapeutics that target cell membrane integrity in combination with inhibitors of DNA synthesis (Michail et al. (2013) Antimicrobial Agents and Chemotherapy 57:6028-6033) or antibiotics that target protein synthesis (Rodriguez-Avial et al. (2015) Int. J. Antimicrob. Agents 46:616-621); and (2) reduced emergence of resistance, i.e., the likelihood of resistance against two drugs is lower than that for an individual therapy (Lee et al. (2009) J. Clin. Microbiol. 47:1611-1612, Khameneh et al. (2016) Microb. Pathog. 95:32-42).

Given the significant risk that antibiotic resistance presents to human and animal health, there is a need to develop novel drug/antibacterial approaches to treat and prevent infection. There is also a need to mitigate the toxicity of our existing drugs of last resort.

The present invention seeks to address these needs by providing compositions and combination products comprising a salicylamide compound and an agent that increases the permeability of bacterial cell membranes.

### SUMMARY OF THE INVENTION

The inventions described and claimed herein have many attributes and embodiments including, but not limited to, those set forth or described or referenced in this Summary of the Invention. It is not intended to be all-inclusive and the inventions described and claimed herein are not limited to or by the features or embodiments identified in this Summary of the Invention, which is included for purposes of illustration only and not restriction.

In one aspect the present invention provides a pharmaceutical composition comprising a salicylamide compound and an agent that increases the permeability of a bacterial cell membrane.

In another aspect the present invention provides a pharmaceutical composition comprising niclosamide and a polymyxin, including polymyxin B or polymyxin E, wherein the polymyxin increases the permeability of a bacterial cell membrane.

In yet another aspect the present invention provides a pharmaceutical composition comprising a compound of Formula I and a polymyxin, including polymyxin B or polymyxin E, wherein the polymyxin increases the permeability of a bacterial cell membrane, and wherein the compound of Formula I is as defined:

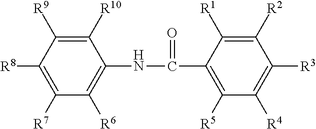

where R₁, R₂, R₃, R₄, R₅, R₆, R₇, R₈, R₉, and R₁₀ are as defined in Table 1.

In yet another aspect the present invention provides a pharmaceutical composition comprising oxyclozanide and a polymyxin, including polymyxin B or polymyxin E, wherein the polymyxin increases the permeability of a bacterial cell membrane.

In yet another aspect the present invention provides a pharmaceutical composition comprising nitazoxanide and a polymyxin, including polymyxin B or polymyxin E, wherein the polymyxin increases the permeability of a bacterial cell membrane.

In yet another aspect the present invention provides a pharmaceutical composition comprising closantel and a polymyxin, including polymyxin B or polymyxin E, wherein the polymyxin increases the permeability of a bacterial cell membrane.

In another aspect the present invention provides a pharmaceutical composition comprising niclosamide and gramicidin, wherein the gramicidin increases the permeability of a bacterial cell membrane.

In yet another aspect the present invention provides a pharmaceutical composition comprising a compound of Formula I and gramicidin, wherein the gramicidin increases the permeability of a bacterial cell membrane, and wherein the compound of Formula I is as defined:

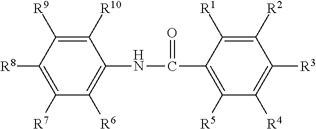

where R₁, R₂, R₃, R₄, R₅, R₆, R₇, R₈, R₉, and R₁₀ are as defined in Table 1.

In yet another aspect the present invention provides a pharmaceutical composition comprising oxyclozanide and gramicidin, wherein the gramicidin increases the permeability of a bacterial cell membrane.

In yet another aspect the present invention provides a pharmaceutical composition comprising nitazoxanide and gramicidin, wherein the gramicidin increases the permeability of a bacterial cell membrane.

In yet another aspect the present invention provides a pharmaceutical composition comprising closantel and gramicidin, wherein the gramicidin increases the permeability of a bacterial cell membrane.

In a further aspect of the present invention there is provided a combination product comprising a salicylamide compound and an agent that increases the permeability of a bacterial cell membrane.

In yet a further aspect of the present invention there is provided a synergistic combination comprising a salicylamide compound and an agent that increases the permeability of a bacterial cell membrane.

In yet a further aspect of the present invention there is provided an anti-bacterial agent comprising a salicylamide compound and an agent that increases the permeability of a bacterial cell membrane.

In yet a further aspect the present invention provides a composition, including a biological composition, comprising a salicylamide compound and an agent that increases the permeability of a bacterial cell membrane.

In yet a further aspect the present invention provides a combination product, a synergistic combination, an anti-bacterial agent or a composition, including a biological composition, comprising niclosamide and a polymyxin, including polymyxin B or polymyxin E, wherein the polymyxin increases the permeability of a bacterial cell membrane.

In yet another aspect the present invention provides a combination product, a synergistic combination, an anti-bacterial agent or a composition, including a biological composition, comprising a compound of Formula I and a polymyxin, including polymyxin B or polymyxin E, wherein the polymyxin increases the permeability of a bacterial cell membrane, and wherein the compound of Formula I is as defined:

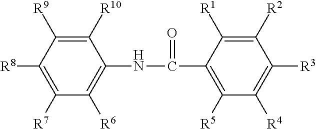

where R₁, R₂, R₃, R₄, R₅, R₆, R₇, R₈, R₉, and R₁₀ are as defined in Table 1.

In yet another aspect the present invention provides a combination product, a synergistic combination, an anti-bacterial agent or a composition, including a biological composition, comprising oxyclozanide and a polymyxin, including polymyxin B or polymyxin E, wherein the polymyxin increases the permeability of a bacterial cell membrane.

In yet another aspect the present invention provides a combination product, a synergistic combination, an anti-bacterial agent or a composition, including a biological composition, comprising nitazoxanide and a polymyxin, including polymyxin B or polymyxin E, wherein the polymyxin increases the permeability of a bacterial cell membrane.

In yet another aspect the present invention provides a combination product, a synergistic combination, an anti-bacterial agent or a composition, including a biological composition, comprising closantel and a polymyxin, including polymyxin B or polymyxin E, wherein the polymyxin increases the permeability of a bacterial cell membrane.

In another aspect the present invention provides a combination product, a synergistic combination, an anti-bacterial agent or a composition, including a biological composition, comprising niclosamide and gramicidin, wherein the gramicidin increases the permeability of a bacterial cell membrane.

In yet another aspect the present invention provides a combination product, a synergistic combination, an anti-bacterial agent or a composition, including a biological composition, comprising a compound of Formula I and gramicidin, wherein the gramicidin increases the permeability of a bacterial cell membrane, and wherein the compound of Formula I is as defined:

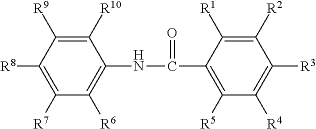

where R₁, R₂, R₃, R₄, R₅, R₆, R₇, R₈, R₉, and R₁₀ are as defined in Table 1.

In yet another aspect the present invention provides a combination product, a synergistic combination, an anti-bacterial agent or a composition, including a biological composition, comprising oxyclozanide and gramicidin, wherein the gramicidin increases the permeability of a bacterial cell membrane.

In yet another aspect the present invention provides a combination product, a synergistic combination, an anti-bacterial agent or a composition, including a biological composition, comprising nitazoxanide and gramicidin, wherein the gramicidin increases the permeability of a bacterial cell membrane.

In yet another aspect the present invention provides a combination product, a synergistic combination, an anti-bacterial agent or a composition, including a biological composition, comprising closantel and gramicidin, wherein the gramicidin increases the permeability of a bacterial cell membrane.

In yet another aspect the present invention provides a pharmaceutical composition as described herein or a combination product, a synergistic combination, an anti-bacterial agent or a composition as described herein for use in:
(i) treating or preventing a bacterial infection in a human or non-human animal; or
(ii) reducing or eliminating formation of a bacterial biofilm
wherein the infection or biofilm comprises one or more Gram negative bacteria.

In yet another aspect the present invention provides a method for treating or preventing a bacterial infection in a patient or for reducing or eliminating formation of a bacterial biofilm comprising administering an antibiotically effective amount of a salicylamide compound and an agent that increases the permeability of a bacterial cell membrane, wherein the infection or biofilm comprises one or more Gram negative bacteria.

In yet a further aspect the present invention provides an article of manufacture comprising package material containing a salicylamide compound and an agent that increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In another aspect the present invention provides an article of manufacture comprising package material containing a niclosamide and a polymyxin, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing a compound of Formula I and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria,

and wherein the compound of Formula I is as defined:

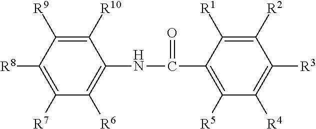

and R₁, R₂, R₃, R₄, R₅, R₆, R₇, R₈, R₉, and R₁₀ are as defined in Table 1.

In yet another aspect the present invention provides an article of manufacture comprising package material containing oxyclozanide and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing nitazoxanide and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing closantel and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In another aspect the present invention provides an article of manufacture comprising package material containing a niclosamide and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing a compound of Formula I and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria,

and wherein the compound of Formula I is as defined:

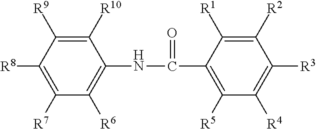

and R₁, R₂, R₃, R₄, R₅, R₆, R₇, R₈, R₉, and R₁₀ are as defined in Table 1.

In yet another aspect the present invention provides an article of manufacture comprising package material containing oxyclozanide and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing nitazoxanide and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing closantel and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

The present invention further contemplates pharmaceutical compositions, combination products, synergistic combinations, anti-bacterial agents, compositions, including biological compositions, and articles of manufacture which exclude niclosamide and colistin.

Accordingly, in another aspect the present invention provides a pharmaceutical composition comprising a salicylamide compound and an agent that increases the permeability of a bacterial cell membrane, provided that the pharmaceutical composition does not contain niclosamide and colistin.

In another aspect the present invention provides a pharmaceutical composition comprising niclosamide and a polymyxin, including polymyxin B or polymyxin E, wherein the polymyxin increases the permeability of a bacterial cell membrane, provided that the polymyxin is not colistin.

In yet another aspect the present invention provides a pharmaceutical composition comprising a compound of Formula I and a polymyxin, including polymyxin B or polymyxin E, wherein the polymyxin increases the permeability of a bacterial cell membrane, and wherein the compound of Formula I is as defined:

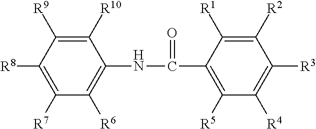

where R₁, R₂, R₃, R₄, R₅, R₆, R₇, R₈, R₉, and R₁₀ are as defined in Table 1, provided that the compound of Formula I is not niclosamide.

In a further aspect of the present invention there is provided a combination product comprising a salicylamide compound and an agent that increases the permeability of a bacterial cell membrane, provided that the combination product does not contain niclosamide and colistin.

In yet a further aspect of the present invention there is provided a synergistic combination comprising a salicylamide compound and an agent that increases the permeability of a bacterial cell membrane, provided that the synergistic combination does not contain niclosamide and colistin.

In yet a further aspect of the present invention there is provided an anti-bacterial agent comprising a salicylamide compound and an agent that increases the permeability of a bacterial cell membrane, provided that the anti-bacterial agent does not contain niclosamide and colistin.

In yet a further aspect the present invention provides a composition, including a biological composition, comprising a salicylamide compound and an agent that increases the permeability of a bacterial cell membrane, provided that the composition does not contain niclosamide and colistin.

In yet a further aspect the present invention provides a combination product, a synergistic combination, an anti-bacterial agent or a composition, including a biological composition, comprising niclosamide and a polymyxin, including polymyxin B or polymyxin E, wherein the polymyxin increases the permeability of a bacterial cell membrane, provided that the polymyxin is not colistin.

In yet another aspect the present invention provides a combination product, a synergistic combination, an anti-bacterial agent or a composition, including a biological composition, comprising a compound of Formula I and a polymyxin, including polymyxin B or polymyxin E, wherein the polymyxin increases the permeability of a bacterial cell membrane, and wherein the compound of Formula I is as defined:

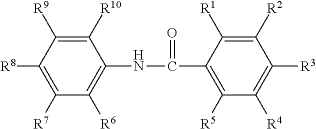

where R₁, R₂, R₃, R₄, R₅, R₆, R₇, R₈, R₉, and R₁₀ are as defined in Table 1, provided that the compound of Formula I is not niclosamide.

In yet another aspect the present invention provides a pharmaceutical composition as described herein or a combination product, a synergistic combination, an anti-bacterial agent or a composition as described herein for use in:
(i) treating or preventing a bacterial infection in a human or non-human animal; or
(ii) reducing or eliminating formation of a bacterial biofilm
wherein the infection or biofilm comprises one or more Gram negative bacteria.

In yet a further aspect the present invention provides an article of manufacture comprising package material containing a salicylamide compound and an agent that increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria, provided that the package material does not contain niclosamide and colistin.

In another aspect the present invention provides an article of manufacture comprising package material containing a niclosamide and a polymyxin, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria, provided that the polymyxin is not colistin.

In yet another aspect the present invention provides an article of manufacture comprising package material containing a compound of Formula I and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria,

and wherein the compound of Formula I is as defined:

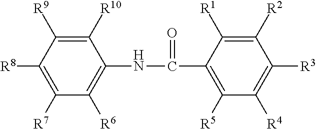

and R₁, R₂, R₃, R₄, R₅, R₆, R₇, R₈, R₉, and R₁₀ are as defined in Table 1, provided that the compound of Formula I is not niclosamide.

The present invention further contemplates methods for treating or preventing a bacterial infection in a patient or for reducing or eliminating formation of a bacterial biofilm by administering antibiotically effective amounts of niclosamide and colistin to the patient or to the biofilm, provided that the bacteria causing infection or biofilm formation is not Klebsiella pneumoniae and/or Acinetobacter baumannii.

Accordingly, in yet another aspect the present invention provides a method for treating or preventing a bacterial infection in a patient or for reducing or eliminating formation of a bacterial biofilm comprising administering to the patient or to the biofilm an antibiotically effective amount of a salicylamide compound and an agent that increases the permeability of a bacterial cell membrane, wherein the infection or biofilm comprises one or more Gram negative bacteria, and wherein when the salicylamide is niclosamide and the agent that increases the permeability of a bacterial cell membrane is colistin, the Gram negative bacteria is not Klebsiella pneumoniae and/or Acinetobacter baumannii.

In yet a further aspect the present invention provides method for treating or preventing a bacterial infection in a patient or for reducing or eliminating formation of a bacterial biofilm comprising administering an antibiotically effective amount of niclosamide and colistin to the patient or to the biofilm, provided that the bacteria causing infection or biofilm formation is not Klebsiella pneumoniae and/or Acinetobacter baumannii.

### BRIEF DESCRIPTION OF THE FIGURES

FIG. 1 shows a heatmap of niclosamide/colistin synergy against E. coli MG1655. This Figure shows percentage growth of E. coli MG1655 in LB amended with niclosamide and colistin as indicated, relative to unchallenged control. Data are the mean of four independent replicates. An overnight culture of E. coli MG1655 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 2 shows a synergy analysis of the effects of niclosamide and colistin against E. coli MG1655. Each cell of each table corresponds to the equivalent cell of the heatmap analysis table in FIG. 1. The right hand panel shows the predicted reduction in percentage turbidity for the averaged microplate data had the effects of niclosamide and colistin been additive. The left hand panel records the actual reduction in measured percentage turbidity, as derived from the data presented in FIG. 1. The highlighted cells in the left hand panel represent concentrations where compound synergy was detected, i.e. the measured percentage reduction in culture turbidity was greater than predicted had the effects of each compound been additive.

FIG. 3 shows a heatmap of niclosamide/polymyxin B synergy against E. coli MG1655. This Figure shows percentage growth of E. coli MG1655 in LB amended with niclosamide and polymyxin B as indicated, relative to unchallenged control. Data are the mean of four independent replicates. An overnight culture of E. coli MG1655 is used to inoculate fresh aliquots of LB media, which is incubated at 30° C., 200 rpm for 2 h. 40 μL aliquots of the culture are subsequently added to individual wells of a multiplex assay containing a 2-fold dilution series of polymyxin B and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 4 shows a synergy analysis of the effects of niclosamide and polymyxin B against E. coli MG1655. Each cell of each table corresponds to the equivalent cell of the heatmap analysis table in FIG. 3. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 3. The highlighted cells in the left hand panel represent concentrations where compound synergy was detected, i.e. the measured percentage reduction in culture turbidity was greater than predicted had the effects of each compound been additive.

FIG. 5 shows a heatmap of niclosamide/colistin synergy against E. coli W3110. This Figure shows percentage growth of E. coli W3110 in LB amended with niclosamide and colistin as indicated, relative to unchallenged control. Data are the mean of two independent replicates each comprising two technical replicates. An overnight culture of E. coli W3110 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 200 rpm for 3 h. Culture turbidity is monitored by optical density at 600 nm in order to calculate percentage growth relative to the 0 μM control for each strain.

FIG. 6 shows a synergy analysis of the effects of niclosamide and colistin against E. coli W3110. Each cell of each table corresponds to the equivalent cell of the heatmap analysis table in FIG. 5. The right hand panel shows the predicted reduction in percentage turbidity for the averaged microplate data had the effects of niclosamide and colistin been additive. The left hand panel records the actual reduction in measured percentage turbidity, as derived from the data presented in FIG. 5. The highlighted cells in the left hand panel represent concentrations where compound synergy was detected, i.e. the measured percentage reduction in culture turbidity was greater than predicted had the effects of each compound been additive.

FIG. 7 shows a heatmap of niclosamide/polymyxin B synergy against E. coli W3110. This Figure shows percentage growth of E. coli W3110 in LB amended with niclosamide and polymyxin B as indicated, relative to unchallenged control. Data are the mean of two independent replicates each comprising two technical replicates. An overnight culture of E. coli W3110 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 3 h. Culture turbidity is monitored by optical density at 600 nm in order to calculate percentage growth relative to the 0 μM control for each strain.

FIG. 8 shows a synergy analysis of the effects of niclosamide and polymyxin B against E. coli W3110. Each cell of each table corresponds to the equivalent cell of the heatmap analysis table in FIG. 7. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 7. The highlighted cells in the left hand panel represent concentrations where compound synergy was detected, i.e. the measured percentage reduction in culture turbidity was greater than predicted had the effects of each compound been additive.

FIG. 9 shows a heatmap of niclosamide/colistin synergy against β-lactam resistant E. coli (NZ isolate ARL06/624). This Figure shows percentage growth of β-lactam resistant E. coli (NZ isolate ARL06/624) in LB amended with niclosamide and colistin as indicated, relative to unchallenged control. Data are the mean of two independent replicates each comprising two technical replicates. An overnight culture of β-lactam resistant E. coli (NZ isolate ARL06/624) is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 200 rpm for 3 h. Culture turbidity is monitored by optical density at 600 nm in order to calculate percentage growth relative to the 0 μM control for each strain.

FIG. 10 shows a synergy analysis of the effects of niclosamide and colistin against β-lactam resistant E. coli (NZ isolate ARL06/624). Each cell of each table corresponds to the equivalent cell of the heatmap analysis table in FIG. 9. The right hand panel shows the predicted reduction in percentage turbidity for the averaged microplate data had the effects of niclosamide and colistin been additive. The left hand panel records the actual reduction in measured percentage turbidity, as derived from the data presented in FIG. 9. The highlighted cells in the left hand panel represent concentrations where compound synergy was detected, i.e. the measured percentage reduction in culture turbidity was greater than predicted had the effects of each compound been additive.

FIG. 11 shows a heatmap of niclosamide/polymyxin B synergy against β-lactam resistant E. coli (NZ isolate ARL06/624). This Figure shows percentage growth of β-lactam resistant E. coli (NZ isolate ARL06/624) in LB amended with niclosamide and polymyxin B as indicated, relative to unchallenged control. Data are the mean of two independent replicates each comprising two technical replicates. An overnight culture of β-lactam resistant E. coli (NZ isolate ARL06/624) is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 3 h. Culture turbidity is monitored by optical density at 600 nm in order to calculate percentage growth relative to the 0 μM control for each strain.

FIG. 12 shows a synergy analysis of the effects of niclosamide and polymyxin B against β-lactam resistant E. coli (NZ isolate ARL06/624). Each cell of each table corresponds to the equivalent cell of the heatmap analysis table in FIG. 11. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 11. The highlighted cells in the left hand panel represent concentrations where compound synergy was detected, i.e. the measured percentage reduction in culture turbidity was greater than predicted had the effects of each compound been additive.

FIG. 13 shows a heatmap of niclosamide/colistin synergy against Pseudomonas aeruginosa PAO1. This Figure shows percentage growth of Pseudomonas aeruginosa PAO1 in LB amended with niclosamide and colistin as indicated, relative to unchallenged control. Data are the mean of four independent replicates. An overnight culture of Pseudomonas aeruginosa PAO1 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 14 shows a synergy analysis of the effects of niclosamide and colistin against Pseudomonas aeruginosa PAO1. Each cell of each table corresponds to the equivalent cell of the heatmap analysis table in FIG. 13. The right hand panel shows the predicted reduction in percentage turbidity for the averaged microplate data had the effects of niclosamide and colistin been additive. The left hand panel records the actual reduction in measured percentage turbidity, as derived from the data presented in FIG. 13. The highlighted cells in the left hand panel represent concentrations where compound synergy was detected, i.e. the measured percentage reduction in culture turbidity was greater than predicted had the effects of each compound been additive.

FIG. 15 shows a heatmap of niclosamide/polymyxin B synergy against Pseudomonas aeruginosa PAO1. This Figure shows percentage growth of Pseudomonas aeruginosa PAO1 in LB amended with niclosamide and polymyxin B as indicated, relative to unchallenged control. Data are the mean of four independent replicates. An overnight culture of Pseudomonas aeruginosa PAO1 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 16 shows a synergy analysis of the effects of niclosamide and polymyxin B against Pseudomonas aeruginosa PAO1. Each cell of each table corresponds to the equivalent cell of the heatmap analysis table in FIG. 15. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 15. The highlighted cells in the left hand panel represent concentrations where compound synergy was detected, i.e. the measured percentage reduction in culture turbidity was greater than predicted had the effects of each compound been additive.

FIG. 17 shows a heatmap of niclosamide/colistin synergy against ceftazidime/piperacillin resistant Pseudomonas aeruginosa (NZ isolate AR 00/537). This Figure shows percentage growth of ceftazidime/piperacillin resistant Pseudomonas aeruginosa (NZ isolate AR 00/537) in LB amended with niclosamide and colistin as indicated, relative to unchallenged control. Data are the mean of two independent replicates each comprising two technical replicates. An overnight culture ceftazidime/piperacillin resistant Pseudomonas aeruginosa (NZ isolate AR 00/537) is used to inoculate fresh aliquots of LB media, which is incubated at 30° C., 200 rpm for 3 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 200 rpm for 4 h. Culture turbidity is monitored by optical density at 600 nm in order to calculate percentage growth relative to the 0 μM control for each strain.

FIG. 18 shows a synergy analysis of the effects of niclosamide and colistin against ceftazidime/piperacillin resistant Pseudomonas aeruginosa (NZ isolate AR 00/537). Each cell of each table corresponds to the equivalent cell of the heatmap analysis table in FIG. 17. The right hand panel shows the predicted reduction in percentage turbidity for the averaged microplate data had the effects of niclosamide and colistin been additive. The left hand panel records the actual reduction in measured percentage turbidity, as derived from the data presented in FIG. 17. The highlighted cells in the left hand panel represent concentrations where compound synergy was detected, i.e. the measured percentage reduction in culture turbidity was greater than predicted had the effects of each compound been additive.

FIG. 19 shows a heatmap of niclosamide/polymyxin B synergy against ceftazidime/piperacillin resistant Pseudomonas aeruginosa (NZ isolate AR 00/537). This Figure shows percentage growth of ceftazidime/piperacillin resistant Pseudomonas aeruginosa (NZ isolate AR 00/537) in LB amended with niclosamide and polymyxin B as indicated, relative to unchallenged control. Data are the mean of two independent replicates each comprising two technical replicates. An overnight culture of ceftazidime/piperacillin resistant Pseudomonas aeruginosa (NZ isolate AR 00/537) is used to inoculate fresh aliquots of LB media, which is incubated at 30° C., 200 rpm for 3 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 20 shows a synergy analysis of the effects of niclosamide and polymyxin B against β-lactam resistant E. coli (NZ isolate ARL06/624). Each cell of each table corresponds to the equivalent cell of the heatmap analysis table in FIG. 19. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 19. The highlighted cells in the left hand panel represent concentrations where compound synergy was detected, i.e. the measured percentage reduction in culture turbidity was greater than predicted had the effects of each compound been additive.

FIG. 21 shows a heatmap of niclosamide/colistin synergy against Klebsiella pneumoniae ATCC BAA-1705. This Figure shows percentage growth of Klebsiella pneumoniae ATCC BAA-1705 in LB amended with niclosamide and colistin as indicated, relative to unchallenged control. Data are the mean of four independent replicates. An overnight culture of Klebsiella pneumoniae ATCC BAA-1705 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 22 shows a synergy analysis of the effects of niclosamide and colistin against Klebsiella pneumoniae ATCC BAA-1705. Each cell of each table corresponds to the equivalent cell of the heatmap analysis table in FIG. 21. The right hand panel shows the predicted reduction in percentage turbidity for the averaged microplate data had the effects of niclosamide and colistin been additive. The left hand panel records the actual reduction in measured percentage turbidity, as derived from the data presented in FIG. 21. The highlighted cells in the left hand panel represent concentrations where compound synergy was detected, i.e. the measured percentage reduction in culture turbidity was greater than predicted had the effects of each compound been additive.

FIG. 23 shows a heatmap of niclosamide/polymyxin B synergy against Klebsiella pneumoniae ATCC BAA-1705. This Figure shows percentage growth of Klebsiella pneumoniae ATCC BAA-1705 in LB amended with niclosamide and polymyxin B as indicated, relative to unchallenged control. Data are the mean of four independent replicates. An overnight culture of Klebsiella pneumoniae ATCC BAA-1705 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 24 shows a synergy analysis of the effects of niclosamide and polymyxin B against Klebsiella pneumoniae ATCC BAA-1705. Each cell of each table corresponds to the equivalent cell of the heatmap analysis table in FIG. 23. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 23. The highlighted cells in the left hand panel represent concentrations where compound synergy was detected, i.e. the measured percentage reduction in culture turbidity was greater than predicted had the effects of each compound been additive.

FIG. 25 shows a heatmap of niclosamide/colistin synergy against β-lactam resistant Klebsiella pneumoniae (NZ isolate NIL 05/26). This Figure shows percentage growth of β-lactam resistant Klebsiella pneumoniae (NZ isolate NIL 05/26) in LB amended with niclosamide and colistin as indicated, relative to unchallenged control. Data are the mean of two independent replicates each comprising two technical replicates. An overnight culture of β-lactam resistant Klebsiella pneumoniae (NZ isolate NIL 05/26) is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 1200 rpm for 2 h. Culture turbidity is monitored by optical density at 600 nm in order to calculate percentage growth relative to the 0 μM control for each strain.

FIG. 26 shows a synergy analysis of the effects of niclosamide and colistin against β-lactam resistant Klebsiella pneumoniae (NZ isolate NIL 05/26). Each cell of each table corresponds to the equivalent cell of the heatmap analysis table in FIG. 25. The right hand panel shows the predicted reduction in percentage turbidity for the averaged microplate data had the effects of niclosamide and colistin been additive. The left hand panel records the actual reduction in measured percentage turbidity, as derived from the data presented in FIG. 25. The highlighted cells in the left hand panel represent concentrations where compound synergy was detected, i.e. the measured percentage reduction in culture turbidity was greater than predicted had the effects of each compound been additive.

FIG. 27 shows a heatmap of niclosamide/polymyxin B synergy against β-lactam resistant Klebsiella pneumoniae (NZ isolate NIL 05/26). This Figure shows percentage growth of β-lactam resistant Klebsiella pneumoniae (NZ isolate NIL 05/26) in LB amended with niclosamide and polymyxin B as indicated, relative to unchallenged control. Data are the mean of two independent replicates each comprising two technical replicates. An overnight culture of β-lactam resistant Klebsiella pneumoniae (NZ isolate NIL 05/26) is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 2 h. Culture turbidity is monitored by optical density at 600 nm in order to calculate percentage growth relative to the 0 μM control for each strain.

FIG. 28 shows a synergy analysis of the effects of niclosamide and polymyxin B against β-lactam resistant Klebsiella pneumoniae (NZ isolate NIL 05/26). Each cell of each table corresponds to the equivalent cell of the heatmap analysis table in FIG. 27. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 27. The highlighted cells in the left hand panel represent concentrations where compound synergy was detected, i.e. the measured percentage reduction in culture turbidity was greater than predicted had the effects of each compound been additive.

FIG. 29 shows a heatmap of niclosamide/colistin synergy against Enterobacter cloacae subsp. cloacae ATCC 13047. This Figure shows percentage growth of Enterobacter cloacae subsp. cloacae ATCC 13047 in LB amended with niclosamide and colistin as indicated, relative to unchallenged control. Data are the mean of four independent replicates. An overnight culture of Enterobacter cloacae subsp. cloacae ATCC 13047 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 30 shows a synergy analysis of the effects of niclosamide and colistin against Enterobacter cloacae subsp. cloacae ATCC 13047. Each cell of each table corresponds to the equivalent cell of the heatmap analysis table in FIG. 29. The right hand panel shows the predicted reduction in percentage turbidity for the averaged microplate data had the effects of niclosamide and colistin been additive. The left hand panel records the actual reduction in measured percentage turbidity, as derived from the data presented in FIG. 29. The highlighted cells in the left hand panel represent concentrations where compound synergy was detected, i.e. the measured percentage reduction in culture turbidity was greater than predicted had the effects of each compound been additive.

FIG. 31 shows a heatmap of niclosamide/polymyxin B synergy against Enterobacter cloacae subsp. cloacae ATCC 13047. This Figure shows percentage growth of Enterobacter cloacae subsp. cloacae ATCC 13047 in LB amended with niclosamide and polymyxin B as indicated, relative to unchallenged control. Data are the mean of four independent replicates. An overnight culture of Enterobacter cloacae subsp. cloacae ATCC 13047 is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 32 shows a synergy analysis of the effects of niclosamide and polymyxin B against Enterobacter cloacae subsp. cloacae ATCC 13047. Each cell of each table corresponds to the equivalent cell of the heatmap analysis table in FIG. 31. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 31. The highlighted cells in the left hand panel represent concentrations where compound synergy was detected, i.e. the measured percentage reduction in culture turbidity was greater than predicted had the effects of each compound been additive.

FIG. 33 shows a heatmap of niclosamide/colistin synergy against Salmonella enterica Serovar Typhimurium (SL1344). This Figure shows percentage growth of Salmonella enterica Serovar Typhimurium (SL1344) in LB amended with niclosamide and colistin as indicated, relative to unchallenged control. Data are the mean of four independent replicates. An overnight culture of Salmonella enterica Serovar Typhimurium (SL1344) is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 34 shows a synergy analysis of the effects of niclosamide and colistin against Salmonella enterica Serovar Typhimurium (SL1344). Each cell of each table corresponds to the equivalent cell of the heatmap analysis table in FIG. 33. The right hand panel shows the predicted reduction in percentage turbidity for the averaged microplate data had the effects of niclosamide and colistin been additive. The left hand panel records the actual reduction in measured percentage turbidity, as derived from the data presented in FIG. 33. The highlighted cells in the left hand panel represent concentrations where compound synergy was detected, i.e. the measured percentage reduction in culture turbidity was greater than predicted had the effects of each compound been additive.

FIG. 35 shows a heatmap of niclosamide/polymyxin B synergy against Salmonella enterica Serovar Typhimurium (SL1344). This Figure shows percentage growth of Salmonella enterica Serovar Typhimurium (SL1344) in LB amended with niclosamide and polymyxin B as indicated, relative to unchallenged control. Data are the mean of four independent replicates. An overnight culture of Salmonella enterica Serovar Typhimurium (SL1344) is used to inoculate fresh aliquots of LB media, which is incubated at 30° C., 200 rpm for 2 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 36 shows a synergy analysis of the effects of niclosamide and polymyxin B against Salmonella enterica Serovar Typhimurium (SL1344). Each cell of each table corresponds to the equivalent cell of the heatmap analysis table in FIG. 35. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 35. The highlighted cells in the left hand panel represent concentrations where compound synergy was detected, i.e. the measured percentage reduction in culture turbidity was greater than predicted had the effects of each compound been additive.

FIG. 37 shows a heatmap of niclosamide/colistin synergy against Acinetobacter baumannii (ATCC type strain 19606). This Figure shows percentage growth of Acinetobacter baumannii (ATCC type strain 19606) in LB amended with niclosamide and colistin as indicated, relative to unchallenged control. Data are the mean of two independent replicates each comprising two technical replicates. An overnight culture of Acinetobacter baumannii (ATCC type strain 19606) is used to inoculate fresh aliquots of LB media, which is incubated at 30° C., 200 rpm for 3 h. 40 μL aliquots of the culture are subsequently added to individual wells of a multiplex assay containing a 2-fold dilution series of niclosamide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 38 shows a synergy analysis of the effects of niclosamide and colistin against Acinetobacter baumannii (ATCC type strain 19606). Each cell of each table corresponds to the equivalent cell of the heatmap analysis table in FIG. 37. The right hand panel shows the predicted reduction in percentage turbidity for the averaged microplate data had the effects of niclosamide and colistin been additive. The left hand panel records the actual reduction in measured percentage turbidity, as derived from the data presented in FIG. 37. The highlighted cells in the left hand panel represent concentrations where compound synergy was detected, i.e. the measured percentage reduction in culture turbidity was greater than predicted had the effects of each compound been additive.

FIG. 39 shows a heatmap of niclosamide/polymyxin B synergy against Acinetobacter baumannii (ATCC type strain 19606). This Figure shows percentage growth of Acinetobacter baumannii (ATCC type strain 19606) in LB amended with niclosamide and polymyxin B as indicated, relative to unchallenged control. Data are the mean of two independent replicates each comprising two technical replicates. An overnight culture of Acinetobacter baumannii (ATCC type strain 19606) is used to inoculate fresh aliquots of LB media, which is incubated at 30° C., 200 rpm for 3 h. 40 μL aliquots of the culture are subsequently added to individual wells of a multiplex assay containing a 2-fold dilution series of niclosamide and polymyxin B as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 40 shows a synergy analysis of the effects of niclosamide and polymyxin B against Acinetobacter baumannii (ATCC type strain 19606). Each cell of each table corresponds to the equivalent cell of the heatmap analysis table in FIG. 39. The right hand panel shows the predicted reduction in percentage turbidity for the averaged microplate data had the effects of niclosamide and polymyxin B been additive. The left hand panel records the actual reduction in measured percentage turbidity, as derived from the data presented in FIG. 39. The highlighted cells in the left hand panel represent concentrations where compound synergy was detected, i.e. the measured percentage reduction in culture turbidity was greater than predicted had the effects of each compound been additive.

FIG. 41 shows a heatmap of niclosamide/gramicidin synergy against E. coli W3110. This Figure shows percentage growth of E. coli W3110 in LB amended with niclosamide and gramicidin as indicated, relative to unchallenged control. Data are the mean of four independent replicates (excluding the 40 μM niclosamide row where data is the mean of two independent replicates). An overnight culture of E. coli W3310 is used to inoculate fresh aliquots of LB media, which is incubated at 30° C., 200 rpm for 2 h. 30 μL aliquots of the culture are subsequently added to individual wells of a multiplex assay containing a 2-fold dilution series of niclosamide and gramicidin as indicated or a 0 μM control in a 384-well microplate. The plate is incubated at 30° C., 200 rpm for 4.5 h. Culture turbidity is monitored by optical density at 600 nm in order to calculate percentage growth relative to the 0 μM control for each strain.

FIG. 42 shows a synergy analysis of the effects of niclosamide and gramicidin against E. coli W3110. Each cell of each table corresponds to the equivalent cell of the heatmap analysis table in FIG. 41. The right hand panel shows the predicted reduction in percentage turbidity for the averaged microplate data had the effects of niclosamide and gramicidin been additive. The left hand panel records the actual reduction in measured percentage turbidity, as derived from the data presented in FIG. 41. The highlighted cells in the left hand panel represent concentrations where compound synergy was detected, i.e. the measured percentage reduction in culture turbidity was greater than predicted had the effects of each compound been additive.

FIG. 43 shows a heatmap of niclosamide/gramicidin synergy against ceftazidime/piperacillin resistant Pseudomonas aeruginosa (NZ isolate AR 00/537). This Figure shows percentage growth of ceftazidime/piperacillin resistant Pseudomonas aeruginosa (NZ isolate AR 00/537) in LB amended with niclosamide and gramicidin as indicated, relative to unchallenged control. Data are the mean of four independent replicates (excluding the 40 μM niclosamide row where data is the mean of two independent replicates). An overnight culture of ceftazidime/piperacillin resistant Pseudomonas aeruginosa (NZ isolate AR 00/537) is used to inoculate fresh aliquots of LB media, which is incubated at 37° C., 200 rpm for 2 h. 30 μL aliquots of the culture are subsequently added to individual wells of a multiplex assay containing a 2-fold dilution series of niclosamide and gramicidin as indicated or a 0 μM control in a 384-well microplate. The plate is incubated at 37° C., 200 rpm for 4.5 h. Culture turbidity is monitored by optical density at 600 nm in order to calculate percentage growth relative to the 0 μM control for each strain.

FIG. 44 shows a synergy analysis of the effects of niclosamide and gramicidin against ceftazidime/piperacillin resistant Pseudomonas aeruginosa (NZ isolate AR 00/537). Each cell of each table corresponds to the equivalent cell of the heatmap analysis table in FIG. 43. The right hand panel shows the predicted reduction in percentage turbidity for the averaged microplate data had the effects of niclosamide and gramicidin been additive. The left hand panel records the actual reduction in measured percentage turbidity, as derived from the data presented in FIG. 43. The highlighted cells in the left hand panel represent concentrations where compound synergy was detected, i.e. the measured percentage reduction in culture turbidity was greater than predicted had the effects of each compound been additive.

FIG. 45 shows a heatmap of oxyclozanide/colistin synergy against E. coli MG1655. This Figure shows percentage growth of E. coli MG1655 in LB amended with oxyclozanide and colistin as indicated, relative to unchallenged control. Data are the mean of four independent replicates. An overnight culture of E. coli MG1655 is used to inoculate fresh aliquots of LB media, which is incubated at 30° C., 200 rpm for 2 h. 40 μL aliquots of the culture are subsequently added to individual wells of a multiplex assay containing a 2-fold dilution series of oxyclozanide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 46 shows a synergy analysis of the effects of oxyclozanide and colistin against E. coli MG1655. Each cell of each table corresponds to the equivalent cell of the heatmap analysis table in FIG. 45. The right hand panel shows the predicted reduction in percentage turbidity for the averaged microplate data had the effects of oxyclozanide and colistin been additive. The left hand panel records the actual reduction in measured percentage turbidity, as derived from the data presented in FIG. 45. The highlighted cells in the left hand panel represent concentrations where compound synergy was detected, i.e. the measured percentage reduction in culture turbidity was greater than predicted had the effects of each compound been additive.

FIG. 47 shows a heatmap of oxyclozanide/colistin synergy against ceftazidime/piperacillin resistant Pseudomonas aeruginosa (NZ isolate AR 00/537). This Figure shows percentage growth of ceftazidime/piperacillin resistant Pseudomonas aeruginosa (NZ isolate AR 00/537) in LB amended with oxyclozanide and colistin as indicated, relative to unchallenged control. Data are the mean of two independent replicates. An overnight culture of ceftazidime/piperacillin resistant Pseudomonas aeruginosa (NZ isolate AR 00/537) is used to inoculate fresh aliquots of LB media, which is incubated at 37° C., 200 rpm for 2 h. 30 μL aliquots of the culture are subsequently added to individual wells of a multiplex assay containing a 2-fold dilution series of oxyclozanide and colistin as indicated or a 0 μM control in a 384-well microplate. The plate is incubated at 37° C., 200 rpm for 4.5 h. Culture turbidity is monitored by optical density at 600 nm in order to calculate percentage growth relative to the 0 μM control for each strain.

FIG. 48 shows a synergy analysis of the effects of oxyclozanide and colistin against ceftazidime/piperacillin resistant Pseudomonas aeruginosa (NZ isolate AR 00/537). Each cell of each table corresponds to the equivalent cell of the heatmap analysis table in FIG. 47. The right hand panel shows the predicted reduction in percentage turbidity for the averaged microplate data had the effects of oxyclozanide and colistin been additive. The left hand panel records the actual reduction in measured percentage turbidity, as derived from the data presented in FIG. 47. The highlighted cells in the left hand panel represent concentrations where compound synergy was detected, i.e. the measured percentage reduction in culture turbidity was greater than predicted had the effects of each compound been additive.

FIG. 49 shows a heatmap of oxyclozanide/colistin synergy against β-lactam resistant Klebsiella pneumoniae (NZ isolate NIL 05/26). This Figure shows percentage growth of β-lactam resistant Klebsiella pneumoniae (NZ isolate NIL 05/26) in LB amended with oxyclozanide and colistin as indicated, relative to unchallenged control. Data are the means of duplicate repeats. An overnight culture of β-lactam resistant Klebsiella pneumoniae (NZ isolate NIL 05/26) is used to inoculate fresh aliquots of LB media, which is incubated at 30° C., 200 rpm for 2 h. 30 μL aliquots of the culture are subsequently added to individual wells of a multiplex assay containing a 2-fold dilution series of oxyclozanide and colistin as indicated or a 0 μM control in a 384-well microplate. The plate is incubated at 30° C., 200 rpm for 4 h. Culture turbidity is monitored by optical density at 600 nm in order to calculate percentage growth relative to the 0 μM control for each strain.

FIG. 50 shows a synergy analysis of the effects of oxyclozanide and colistin against β-lactam resistant Klebsiella pneumoniae (NZ isolate NIL 05/26). Each cell of each table corresponds to the equivalent cell of the heatmap analysis table in FIG. 49. The right hand panel shows the predicted reduction in percentage turbidity for the averaged microplate data had the effects of oxyclozanide and colistin been additive. The left hand panel records the actual reduction in measured percentage turbidity, as derived from the data presented in FIG. 49. The highlighted cells in the left hand panel represent concentrations where compound synergy was detected, i.e. the measured percentage reduction in culture turbidity was greater than predicted had the effects of each compound been additive.

FIG. 51 shows a heatmap of oxyclozanide/colistin synergy against Acinetobacter baumannii (ATCC type strain 19606). This Figure shows percentage growth of Acinetobacter baumannii (ATCC type strain 19606) in LB amended with oxyclozanide and colistin as indicated, relative to unchallenged control. Data are the means of duplicate repeats. An overnight culture of Acinetobacter baumannii (ATCC type strain 19606) is used to inoculate fresh aliquots of LB media, which is incubated at 30° C., 200 rpm for 2 h. 30 μL aliquots of the culture are subsequently added to individual wells of a multiplex assay containing a 2-fold dilution series of oxyclozanide and colistin as indicated or a 0 μM control in a 384-well microplate. The plate is incubated at 30° C., 200 rpm for 4 h. Culture turbidity is monitored by optical density at 600 nm in order to calculate percentage growth relative to the 0 μM control for each strain.

FIG. 52 shows a synergy analysis of the effects of oxyclozanide and colistin against Acinetobacter baumannii (ATCC type strain 19606). Each cell of each table corresponds to the equivalent cell of the heatmap analysis table in FIG. 51. The right hand panel shows the predicted reduction in percentage turbidity for the averaged microplate data had the effects of oxyclozanide and colistin been additive. The left hand panel records the actual reduction in measured percentage turbidity, as derived from the data presented in FIG. 51. The highlighted cells in the left hand panel represent concentrations where compound synergy was detected, i.e. the measured percentage reduction in culture turbidity was greater than predicted had the effects of each compound been additive.

FIG. 53 shows a heatmap of nitazoxanide/colistin synergy against E. coli MG1655. This Figure shows percentage growth of E. coli MG1655 in LB amended with nitazoxanide and colistin as indicated, relative to unchallenged control. Data are the mean of four independent replicates. An overnight culture of E. coli MG1655 is used to inoculate fresh aliquots of LB media, which is incubated at 30° C., 200 rpm for 2 h. 40 μL aliquots of the culture are subsequently added to individual wells of a multiplex assay containing a 2-fold dilution series of nitazoxanide and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 54 shows a synergy analysis of the effects of nitazoxanide and colistin against E. coli MG1655. Each cell of each table corresponds to the equivalent cell of the heatmap analysis table in FIG. 53. The right hand panel shows the predicted reduction in percentage turbidity for the averaged microplate data had the effects of nitazoxanide and colistin been additive. The left hand panel records the actual reduction in measured percentage turbidity, as derived from the data presented in FIG. 53. The highlighted cells in the left hand panel represent concentrations where compound synergy was detected, i.e. the measured percentage reduction in culture turbidity was greater than predicted had the effects of each compound been additive.

FIG. 55 shows a heatmap of closantel/colistin synergy against E. coli MG1655. This Figure shows percentage growth of E. coli MG1655 in LB amended with closantel and colistin as indicated, relative to unchallenged control. Data are the mean of four independent replicates. An overnight culture of E. coli MG1655 is used to inoculate fresh aliquots of LB media, which is incubated at 30° C., 200 rpm for 2 h. 40 μL aliquots of the culture are subsequently added to individual wells of a multiplex assay containing a 2-fold dilution series of closantel and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 56 shows a synergy analysis of the effects of closantel and colistin against E. coli MG1655. Each cell of each table corresponds to the equivalent cell of the heatmap analysis table in FIG. 55. The right hand panel shows the predicted reduction in percentage turbidity for the averaged microplate data had the effects of closantel and colistin been additive. The left hand panel records the actual reduction in measured percentage turbidity, as derived from the data presented in FIG. 55. The highlighted cells in the left hand panel represent concentrations where compound synergy was detected, i.e. the measured percentage reduction in culture turbidity was greater than predicted had the effects of each compound been additive.

FIG. 57 shows a heatmap of 2,4-dinitrophenol/colistin synergy against E. coli MG1655. This Figure shows percentage growth of E. coli MG1655 in LB amended with 2,4-dinitrophenol and colistin as indicated, relative to unchallenged control. Data are the mean of four independent replicates. An overnight culture of E. coli MG1655 is used to inoculate fresh aliquots of LB media, which is incubated at 30° C., 200 rpm for 2 h. 40 μL aliquots of the culture are subsequently added to individual wells of a multiplex assay containing a 2-fold dilution series of 2,4-dinitrophenol and colistin as indicated or a 0 μM control in a 384-well microplate. The plate is incubated at 30° C., 1200 rpm for 4 h. Culture turbidity is monitored by optical density at 600 nm in order to calculate percentage growth relative to the 0 μM control for each strain.

FIG. 58 shows a synergy analysis of the effects of 2,4-dinitrophenol and colistin against E. coli MG1655. Each cell of each table corresponds to the equivalent cell of the heatmap analysis table in FIG. 57. The right hand panel shows the predicted reduction in percentage turbidity for the averaged microplate data had the effects of 2,4-dinitrophenol and colistin been additive. The left hand panel records the actual reduction in measured percentage turbidity, as derived from the data presented in FIG. 57. The highlighted cells in the left hand panel represent concentrations where compound synergy was detected, i.e. the measured percentage reduction in culture turbidity was greater than predicted had the effects of each compound been additive.

FIG. 59 shows the intracellular oxidative stress analysis of E. coli strains after challenge with niclosamide as measured via redox sensitive GFP. Panel A depicts intracellular oxidative stress response 7KO:roGFP and 7TL:roGFP after challenge with 10 mM H₂O₂ or 1 mM DTT (to obtain fully oxidized or fully reduced redox signals), or 200 nM, 1 μM, or 10 μM of niclosamide. Panels B and C show the intrabacterial redox potential of 7KOΔtolC:roGFP and 7KO:roGFP strains in response to niclosamide challenge measured using an AMNIS ImageStream system analysing at least 15,000 individual cells per time point and test condition. Cells were fixed with N-ethylmaleimide at various time points post niclosamide incubation, and then passed through a flow cytometry system. The ImageStream recorded each individual cell and all images were processed automatically. The redox potential (405/480 nm ratio) was obtained for each experiment, and together these results created a “redox stress” timeline comparing the niclosamide response in 7KO:roGFP and 7KOΔtolC:roGFP strains (Panel B). Panel C depicts representative microscopy images; in 7KO:roGFP, intracellular bacteria did not experience significant redox stress after niclosamide challenge (Panel C, “wild type”), whereas 7KOΔtolC:roGFP (Panel C, “tolC”) experienced increasing redox stress over the time course of the experiment.

FIG. 60 shows the intracellular oxidative stress analysis of E. coli strains after challenge with niclosamide as measured via redox sensitive GFP. Panel A depicts intracellular oxidative stress response of 7KO:roGFP after challenge with 10 mM H₂O₂ or 1 mM DTT (to obtain fully oxidized or fully reduced redox signals), or 1 μM niclosamide. Panel B shows the intracellular oxidative stress response of 7KO:roGFP in response to 10 mM H₂O₂, or 1 μM niclosamide, or 25 μM PABN, or a combination of 25 μM PABN and 1 μM niclosamide. Panel C shows the intracellular oxidative stress response of 7KO:roGFP in response to 10 mM H₂O₂, or 1 μM niclosamide, or 0.5 μM colistin, or a combination of 0.5 μM colistin and 1 μM niclosamide. Panel D shows the intracellular oxidative stress response of 7KO:roGFP in response to 10 mM H₂O₂, or 1 μM niclosamide, or 0.5 μM polymyxin B (labelled as “polymixin”), or a combination of 0.5 μM polymyxin B and 1 μM niclosamide.

### SELECTED DEFINITIONS

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used in this specification, the words “comprises”, “comprising”, and similar words, are not to be interpreted in an exclusive or exhaustive sense. In other words, they are intended to mean “including, but not limited to”.

As used in this specification, the term “salicylamide compound” includes all salicylamide and salicylanilide compounds as well as derivatives and analogues thereof. Examples of suitable derivatives and analogues of salicylamide compounds are described in further detail below.

As used in this specification, the term “salicylanilide compound” comprises all compounds that are amides of salicylic acid and of aniline, and may therefore be categorized both as a salicylamides and anilides. The term “salicylanilide compound” includes all salicylanilide compounds as well as derivatives and analogues thereof. Examples of suitable derivatives and analogues of salicylanilide compounds are described in further detail below.

As used in this specification, the term “an agent that increases the permeability of a bacterial cell membrane” includes any pharmaceutically or biologically active agent capable of disrupting the cell membrane of bacteria. Examples of agent that increases the permeability of a bacterial cell membrane according to the present invention include, but are not limited to, polymyxins including polymyxin B and polymyxin E (i.e. colistin).

As used in this specification, the terms “increases the permeability”, “increased permeability” and the like are defined as having a property of allowing an increased amount of a drug, such as an antibiotic, to travel through a cellular barrier (e.g. cell membrane or cell junction) relative to a cellular barrier that has not been exposed to the agent.

### DETAILED DESCRIPTION

The present invention is predicated on the surprising and unexpected discovery that specific combinations of drug-based agents which increase the permeability of bacterial cell membranes can potentiate the effect of certain antibiotics, such as (e.g.) salicylamides including niclosamide, nitazoxanide, oxyclozanide and closantel, which when combined demonstrate bactericidal activity against Gram negative bacteria.

This finding is completely unexpected on two levels. Firstly, salicylamides, including niclosamide, are not known to be effective antibiotics against Gram negative bacteria. Second, as recent as 2015, it was reported in the literature that drug combinations comprising conventional antibiotics and membrane-permeabilizing antimicrobial peptides lack synergistic interaction (He et al. (2015) Biochimica et Biophysica Acta 1848:8-15).

Importantly, Applicants demonstrate here that administration of therapeutic combinations is effective against many clinically relevant drug resistant bacteria.

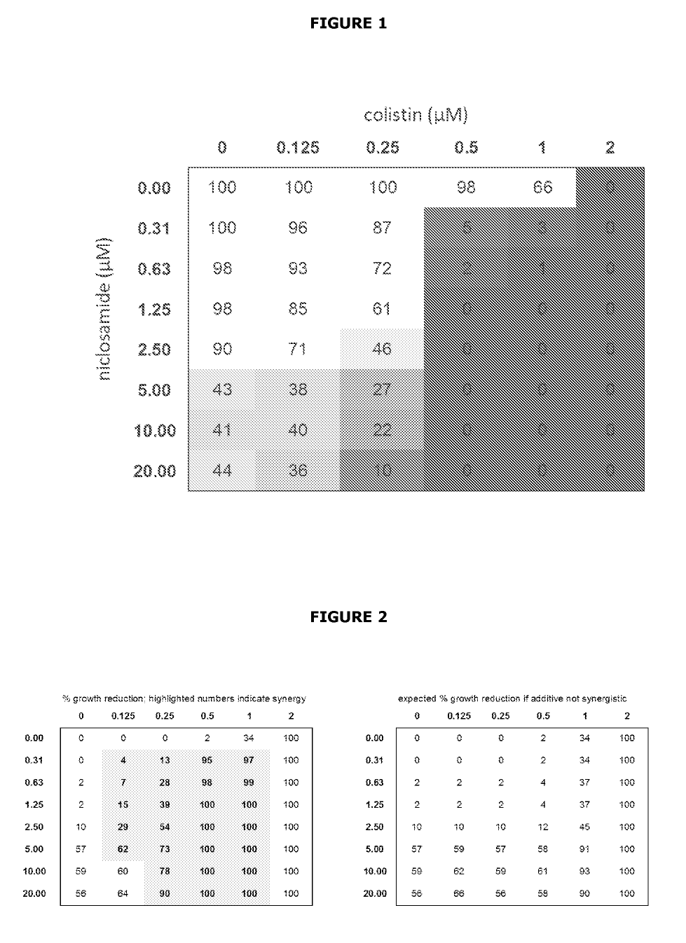

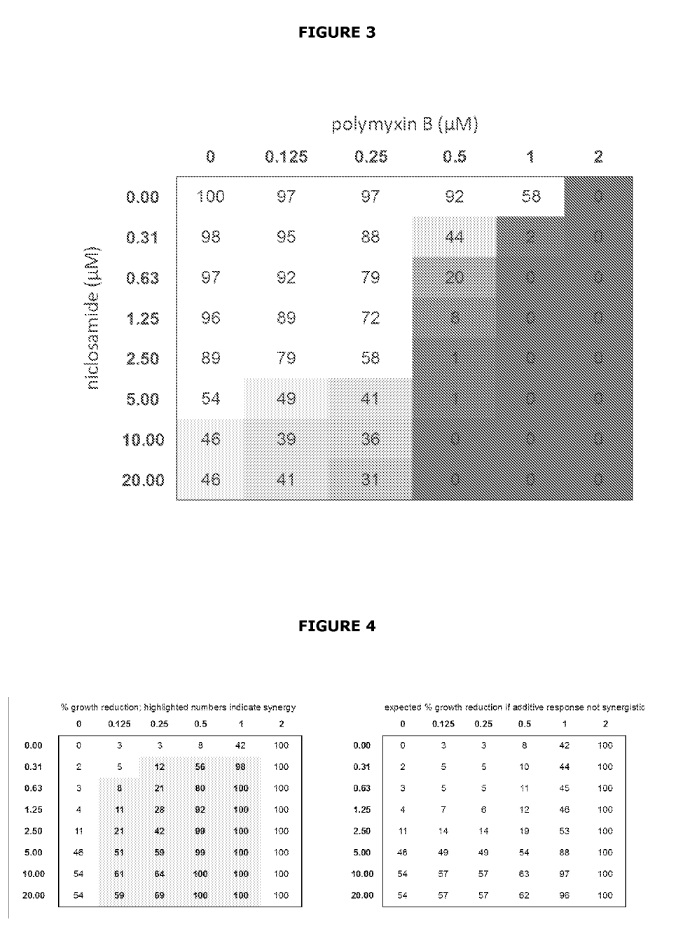

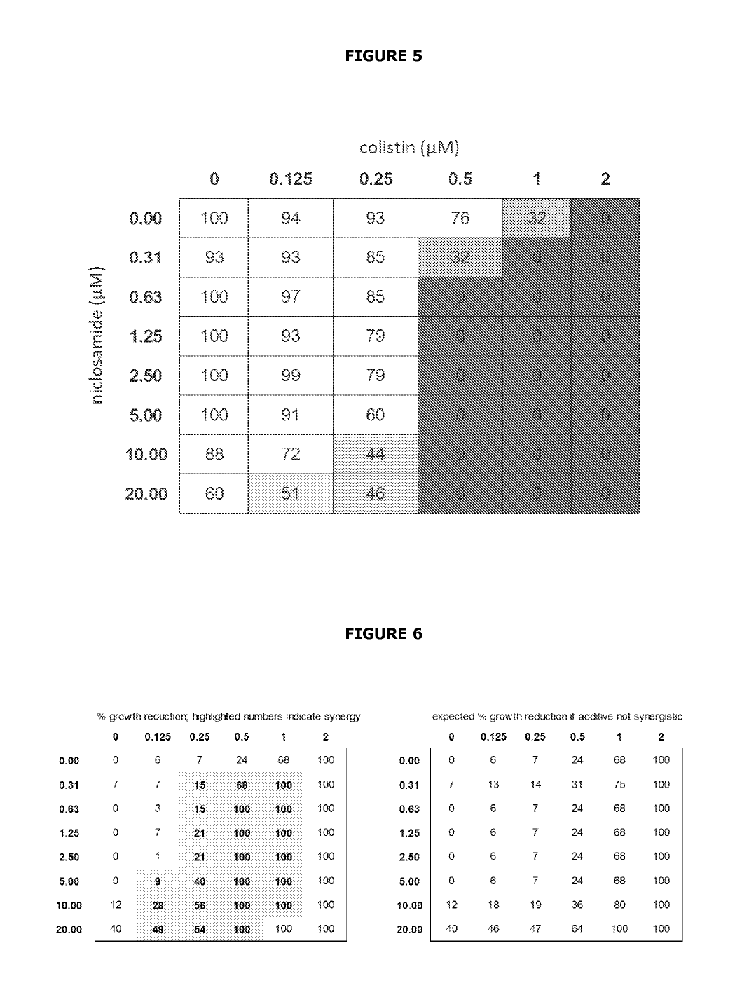

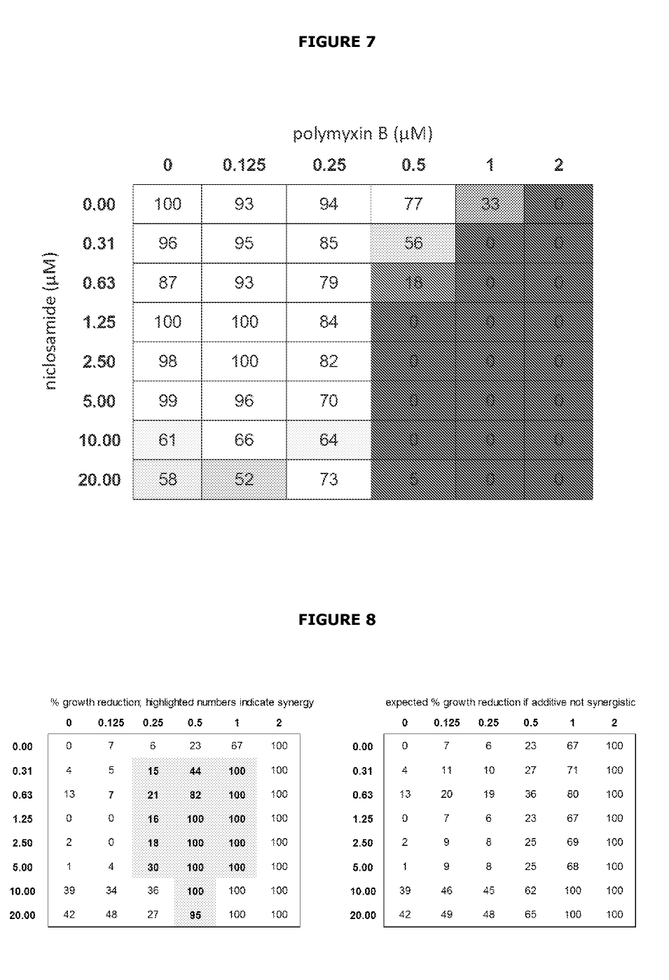

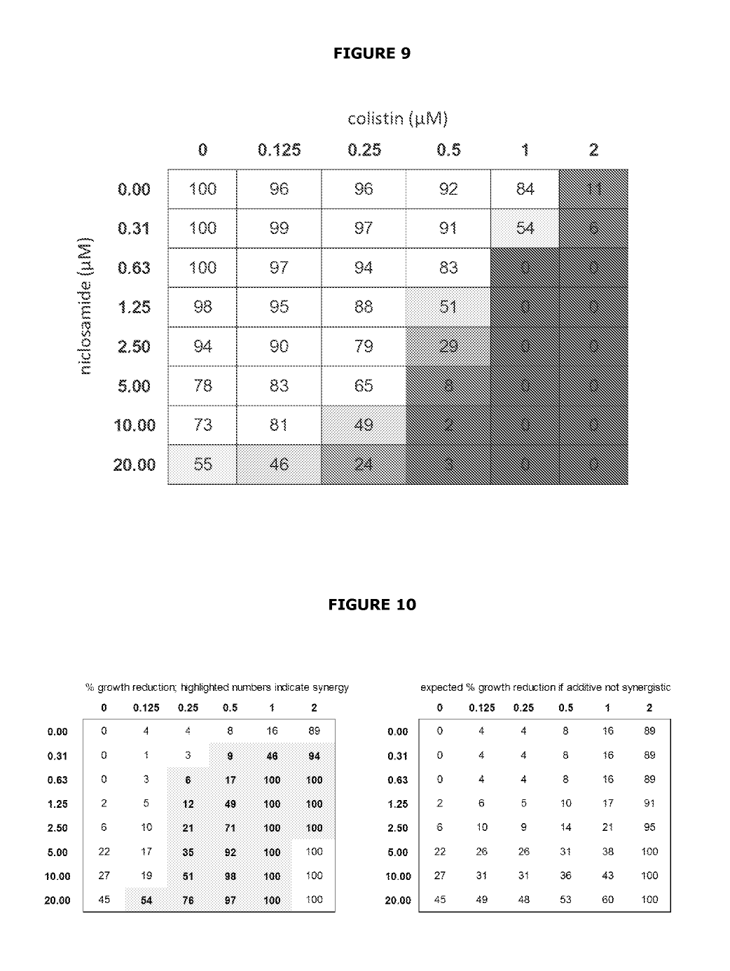

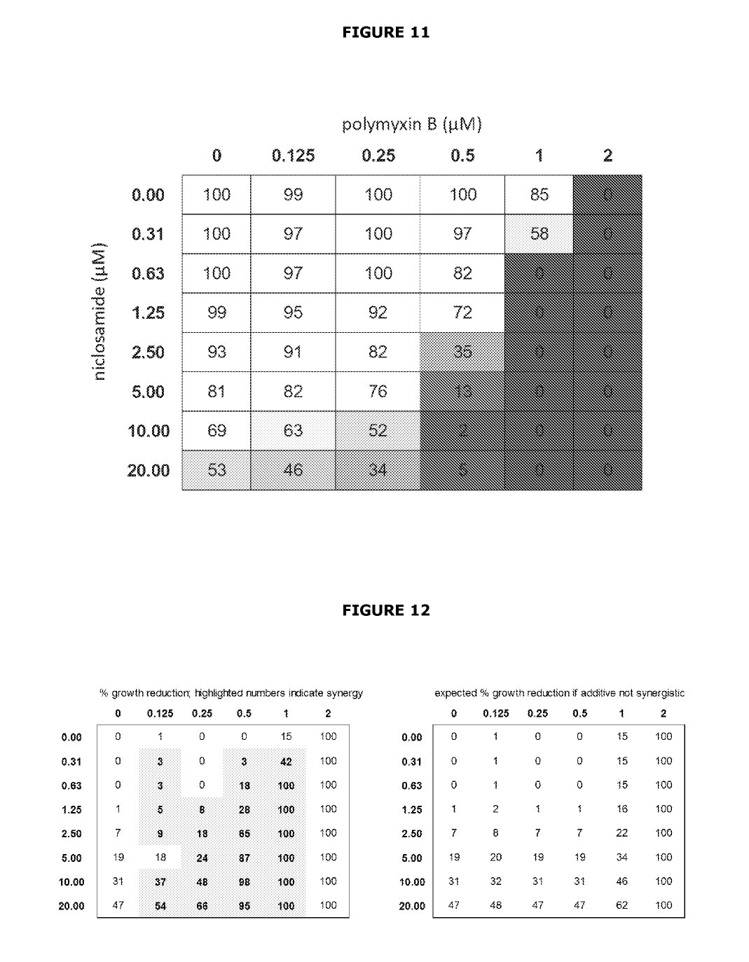

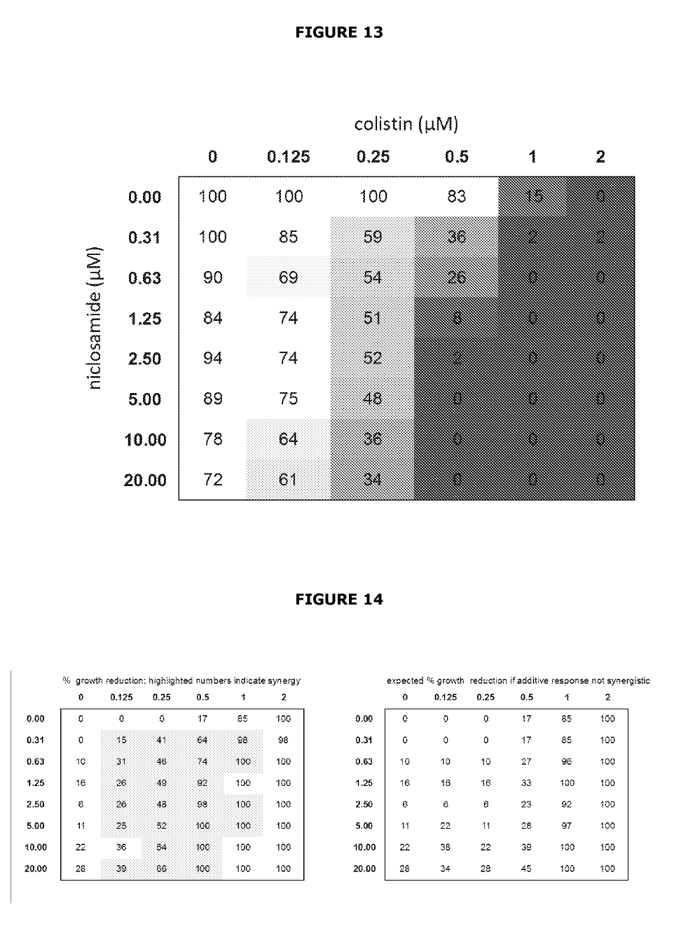

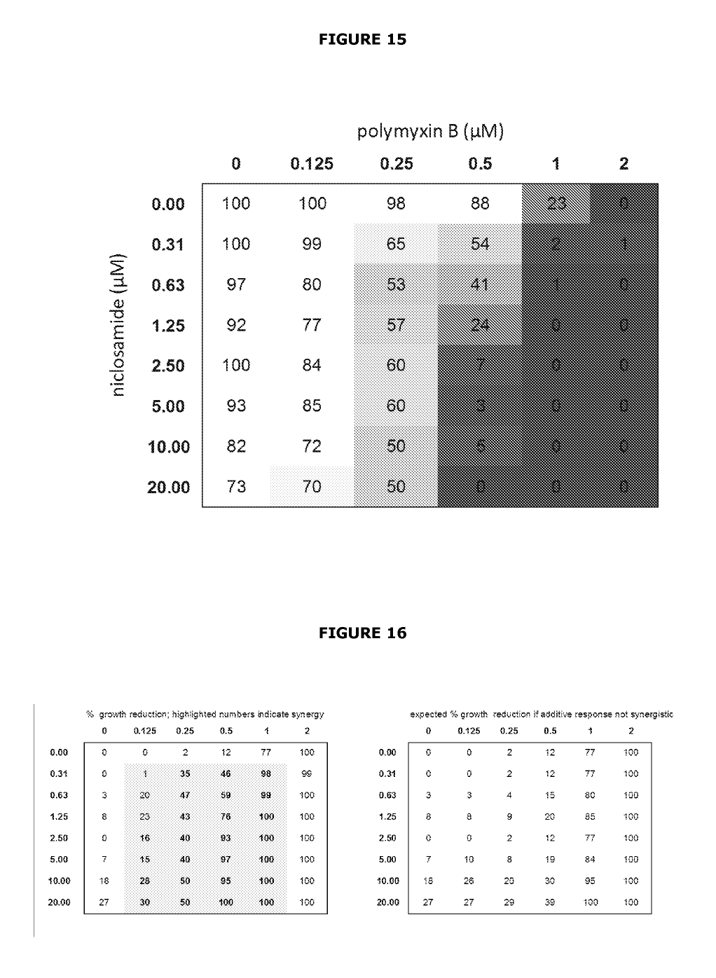

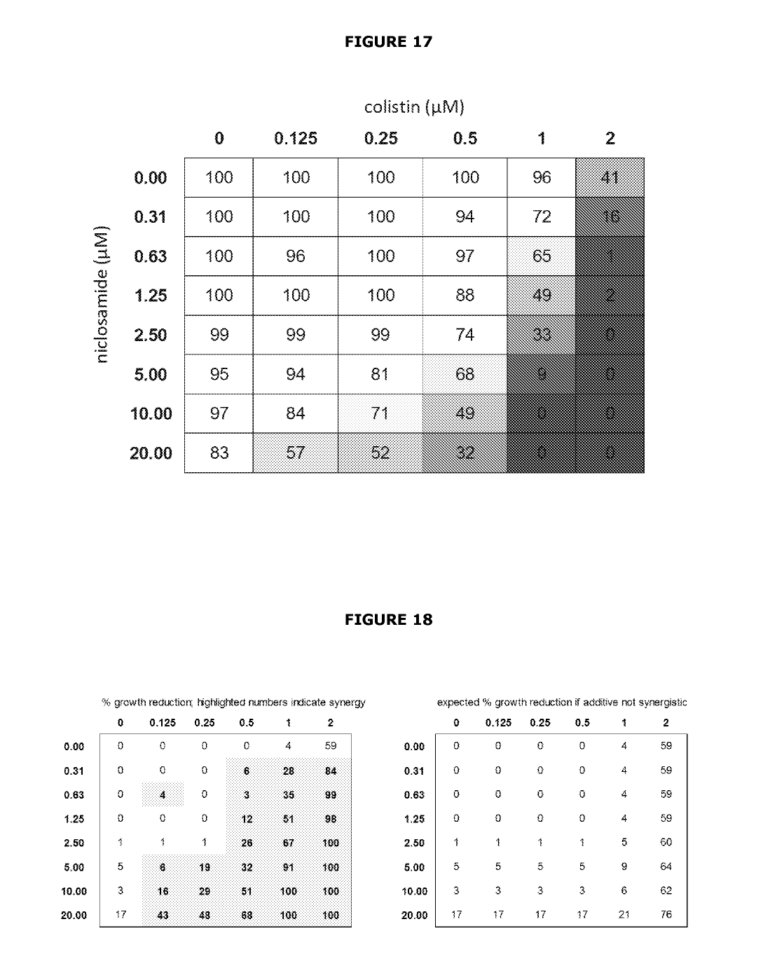

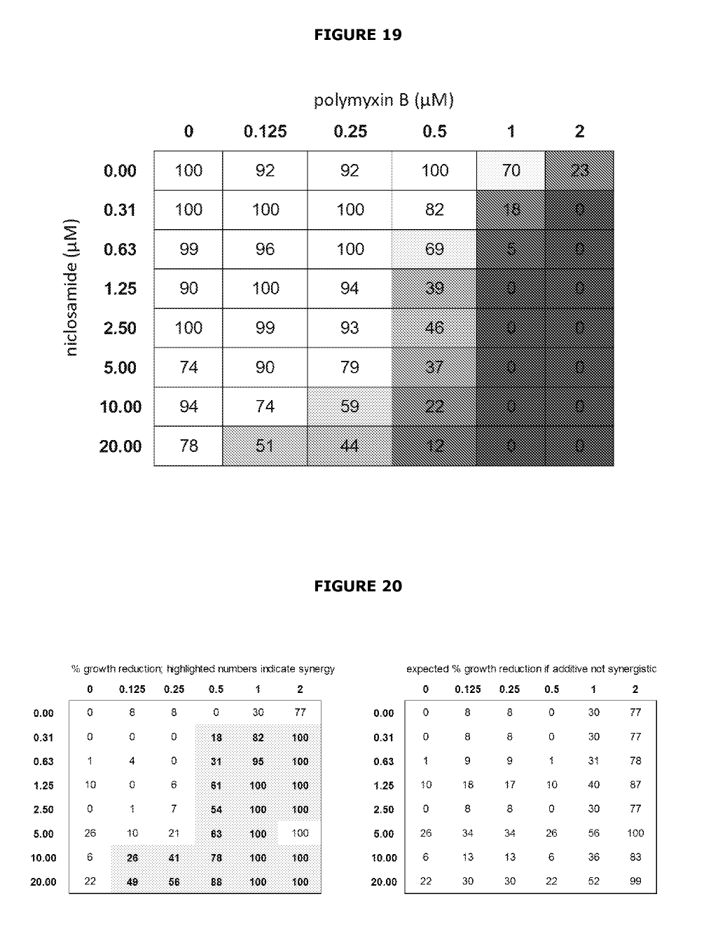

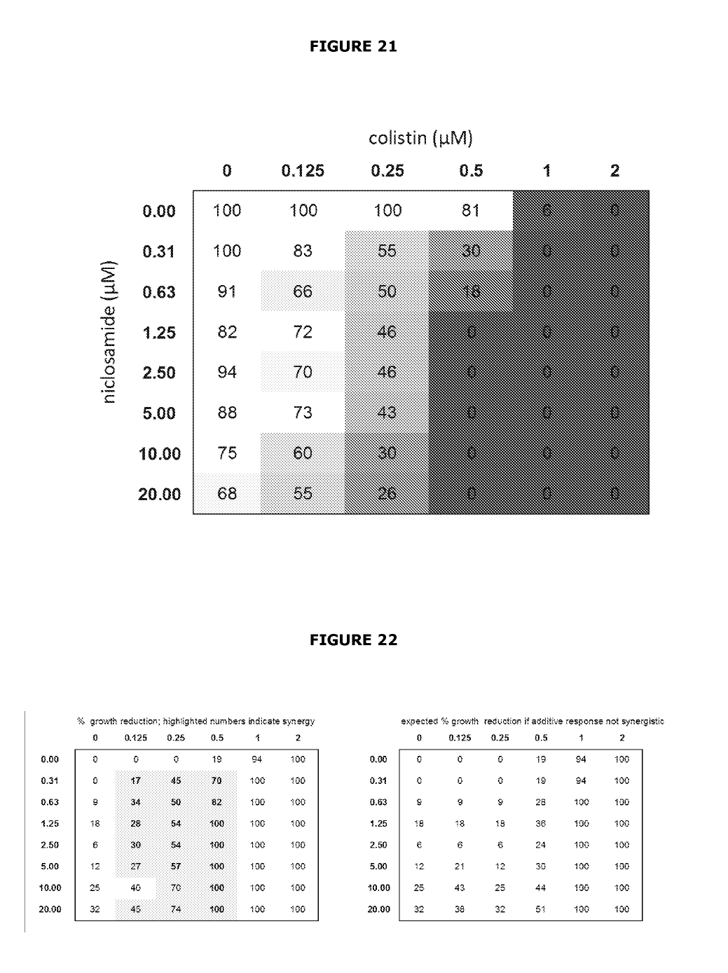

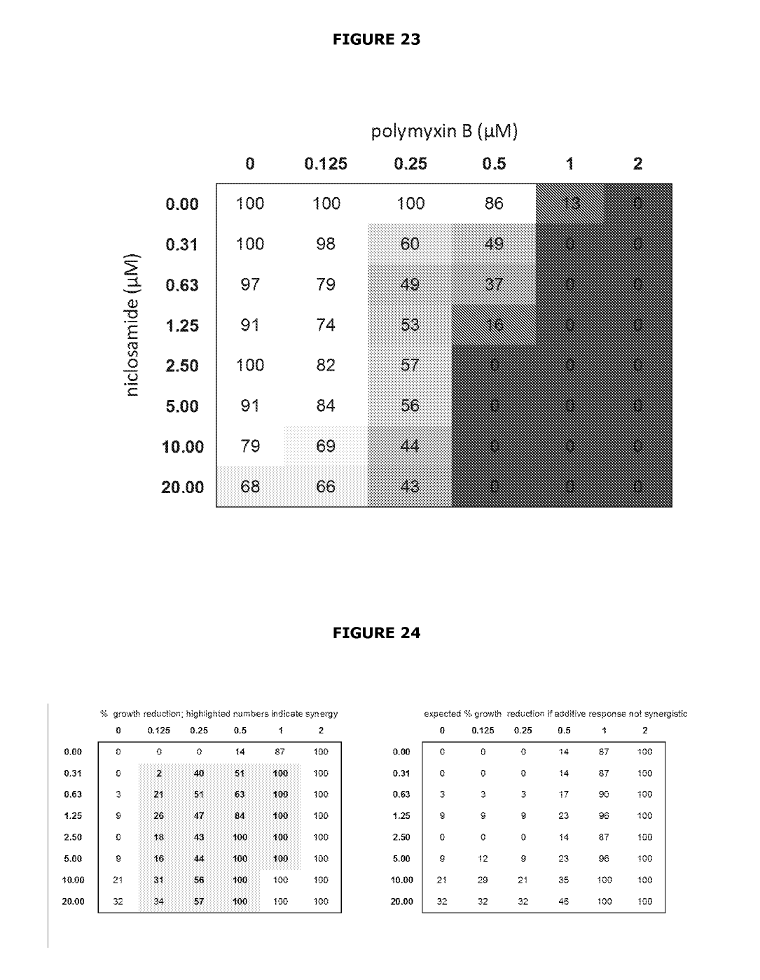

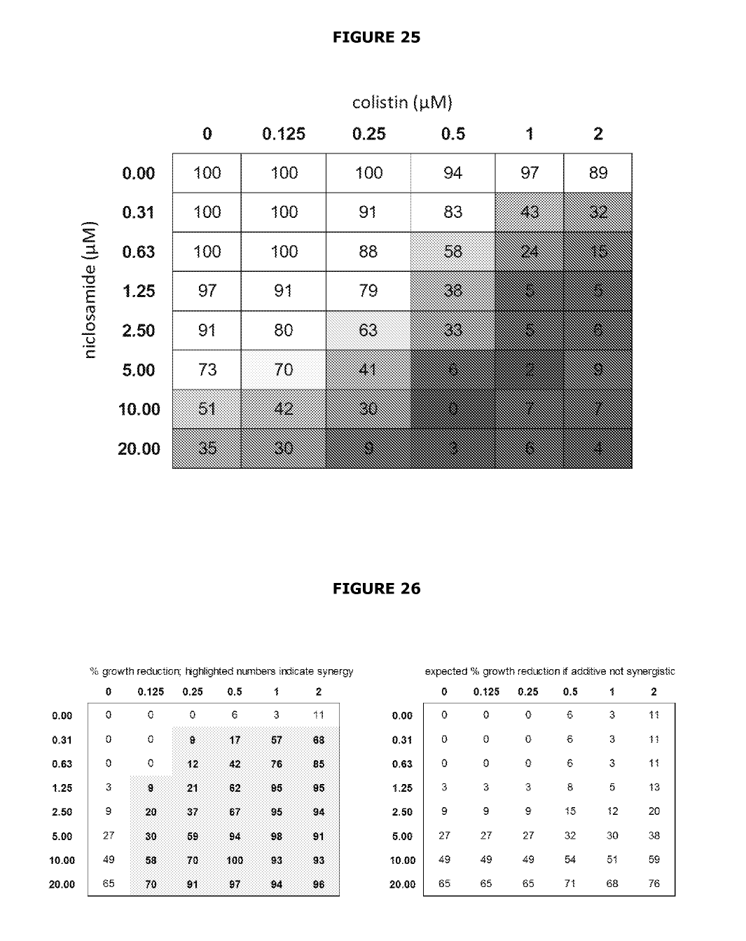

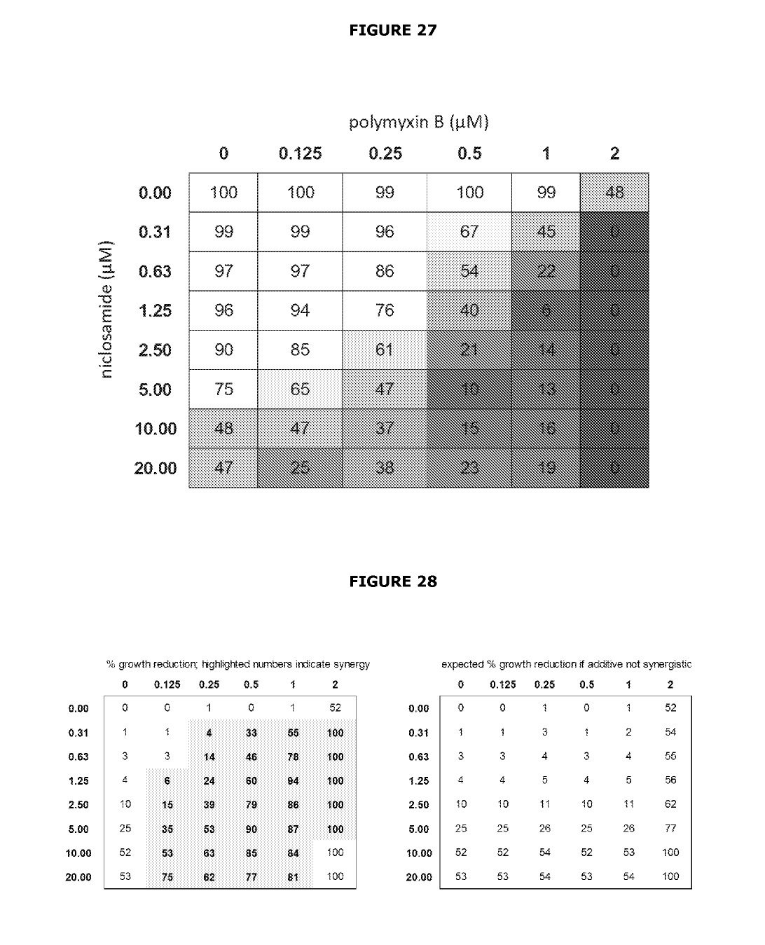

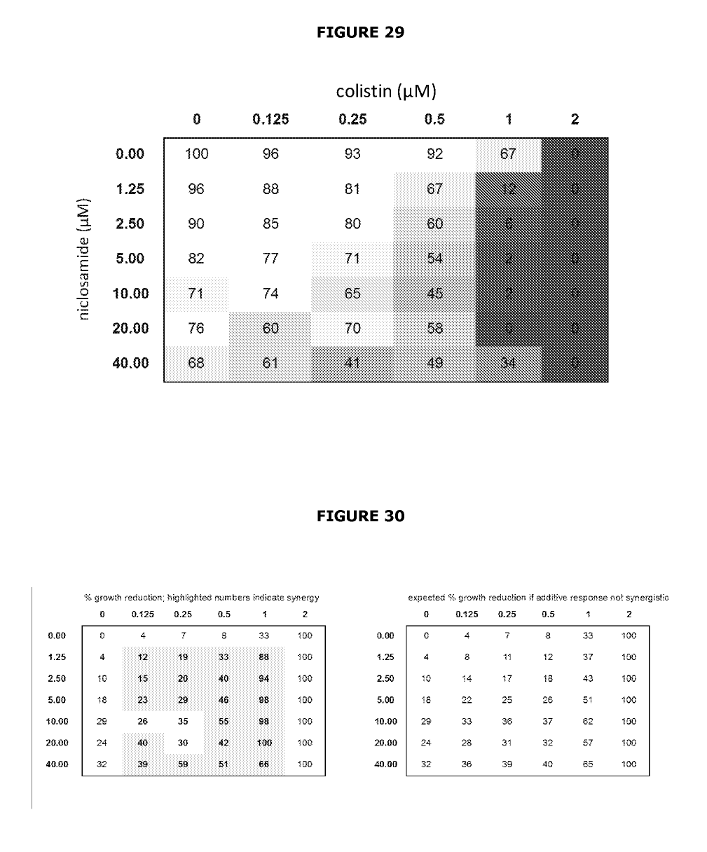

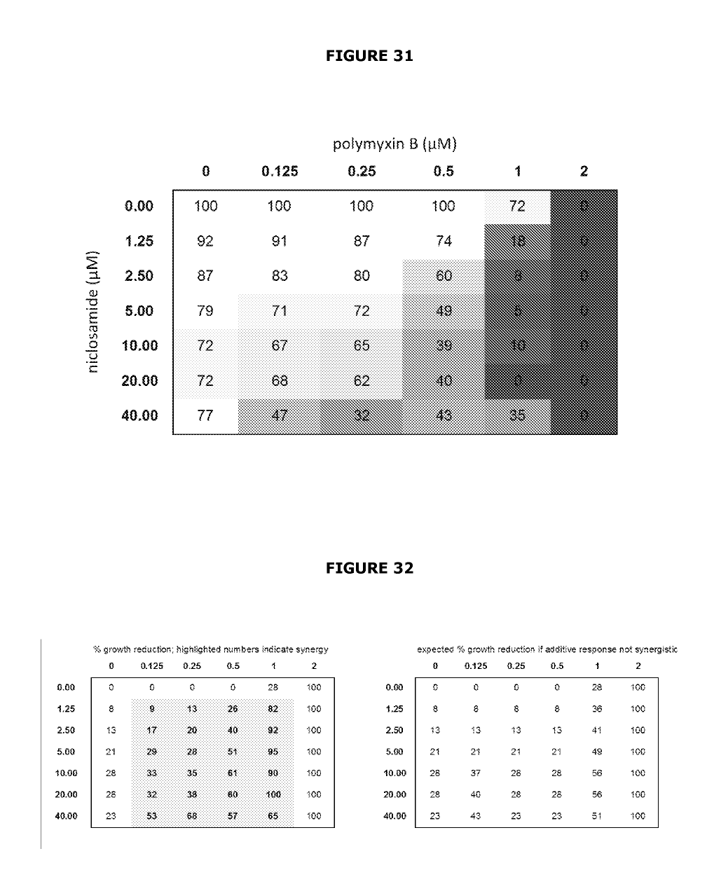

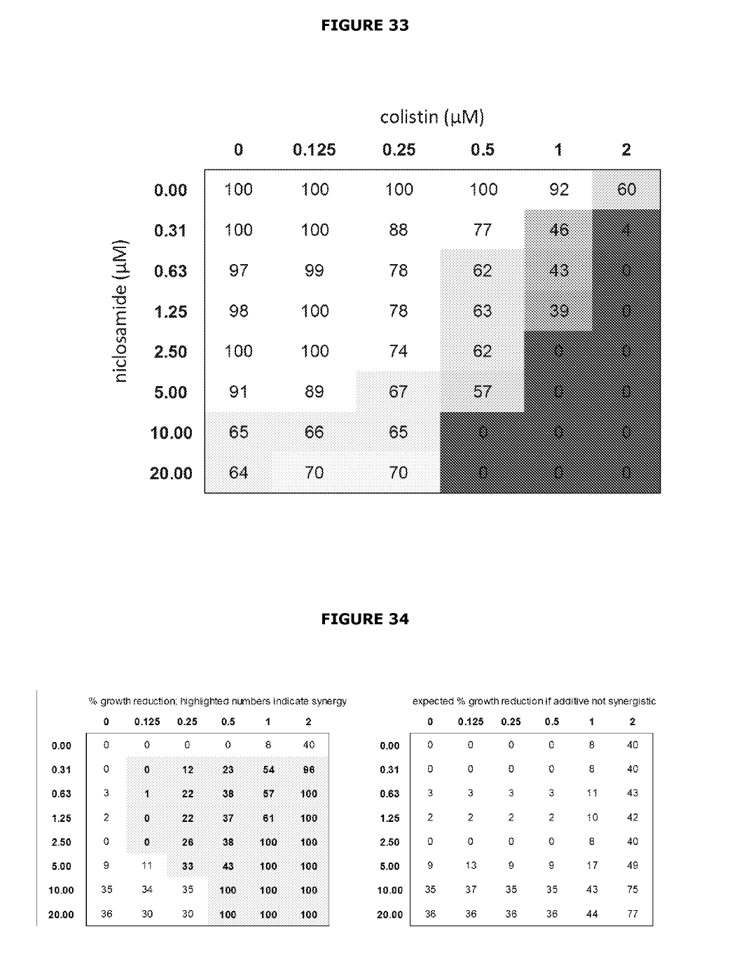

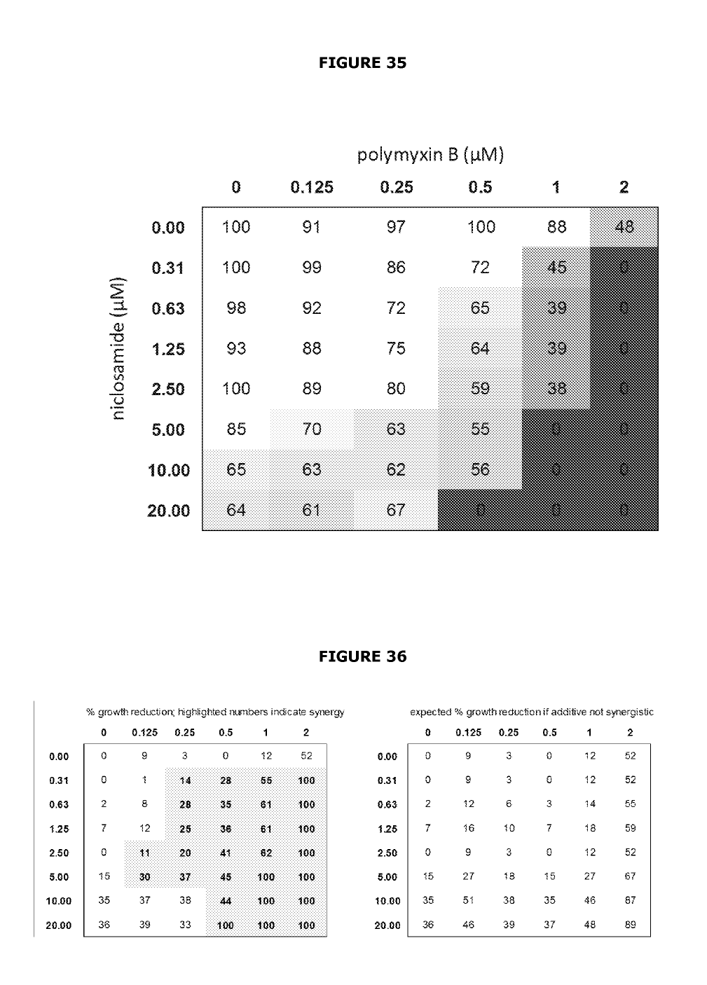

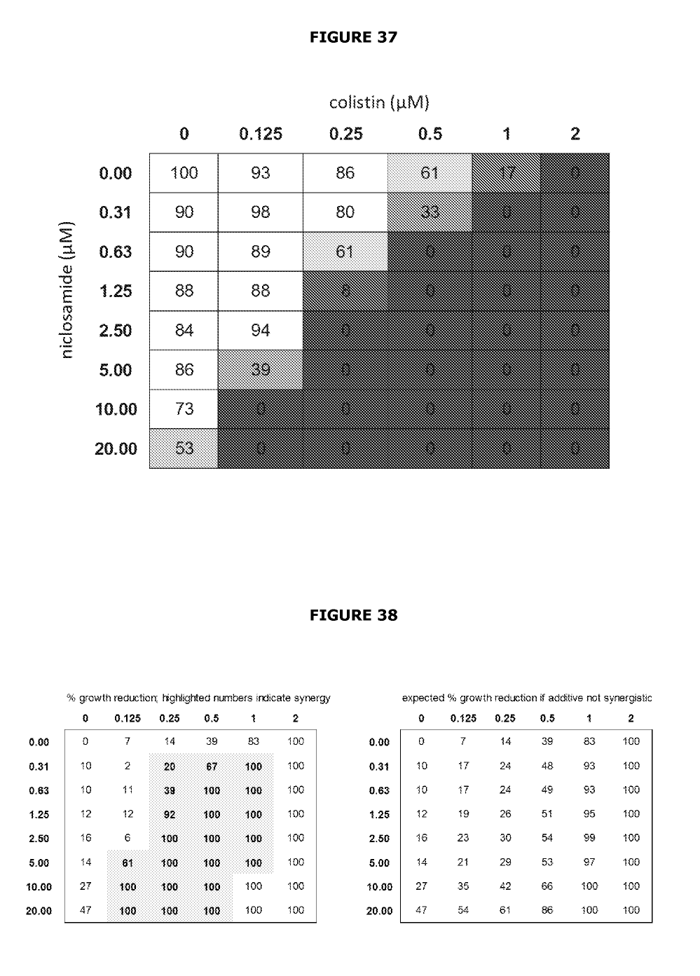

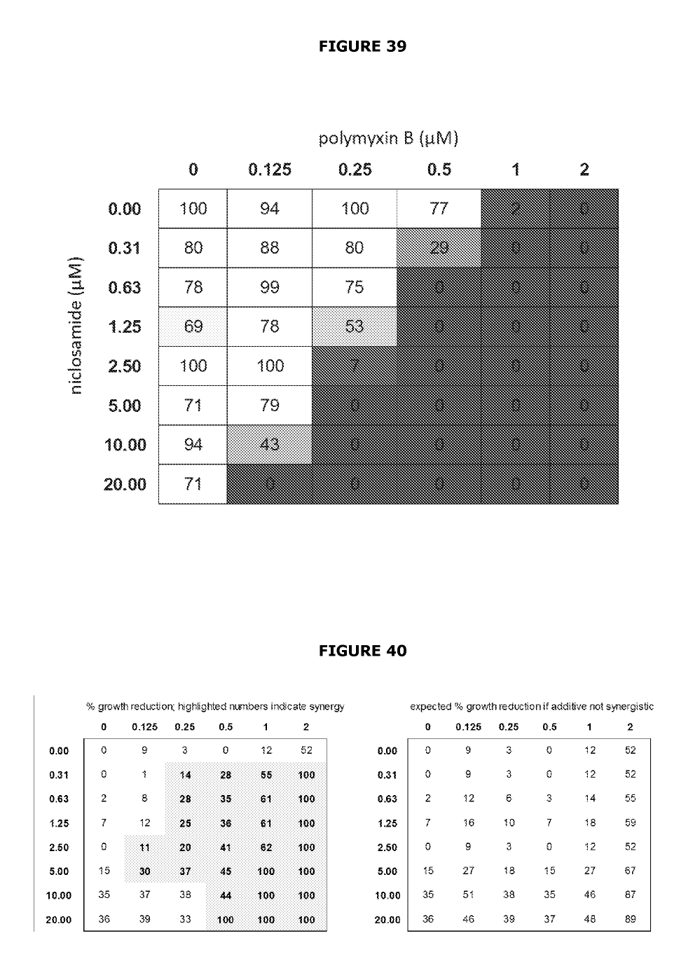

Specifically, niclosamide when used in conjunction with a polymyxin, such as (e.g.) polymyxin B or colistin provides a synergistic growth inhibition effect on a range of different Gram negative bacteria including, but not limited to, Escherichia coli, Pseudomonas aeruginosa, Enterobacter cloacae, Klebsiella pneumonia, Salmonella enterica and Acintetobacter baumannii (FIGS. 1-40). Advantageously, niclosamide is known to be tolerated in humans at high doses, and the Applicants' work also demonstrates that it is an effective antibiotic against Gram negative bacteria, applied in combination with an agent that increases the permeability of the bacterial cell membrane such as colistin or polymyxin B.

Because both niclosamide and compounds that target cell membrane integrity, such as colistin, are FDA-approved drugs, their route to the clinic may be expedited with reduced cost.

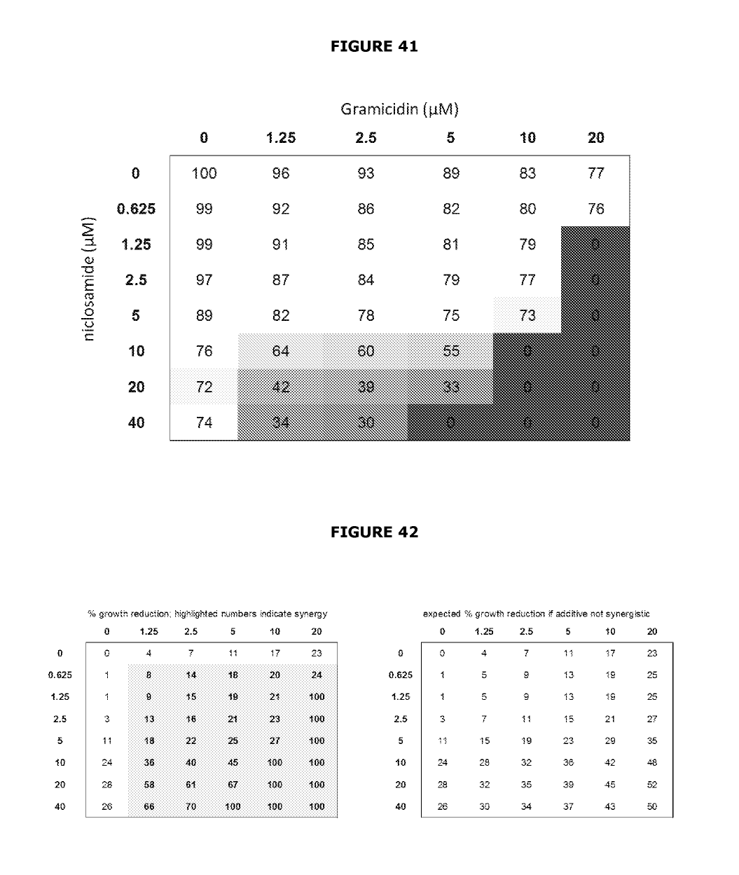

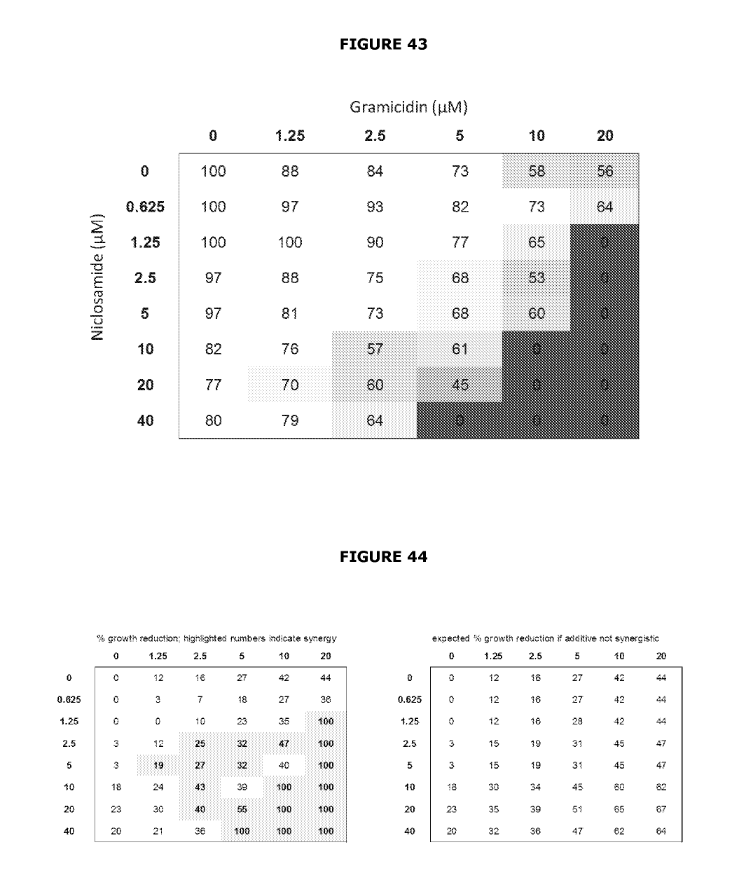

The generic ability of membrane permeabilizing drugs beyond the polymyxin family to sensitize Gram negative bacteria to the antibiotic effects of salicylamide drugs was further demonstrated using the membrane-permeabilizing antibiotic gramicidin (a mixture of gramicidin A, B, C and D; catalog# G5002 from Sigma-Aldrich). Gramicidin provides a synergistic growth inhibition effect on a range of different Gram negative bacteria including, but not limited to, E. coli laboratory strain W3110 and an antibiotic-resistant clinical isolate of Pseudomonas aeruginosa (FIGS. 41-44).

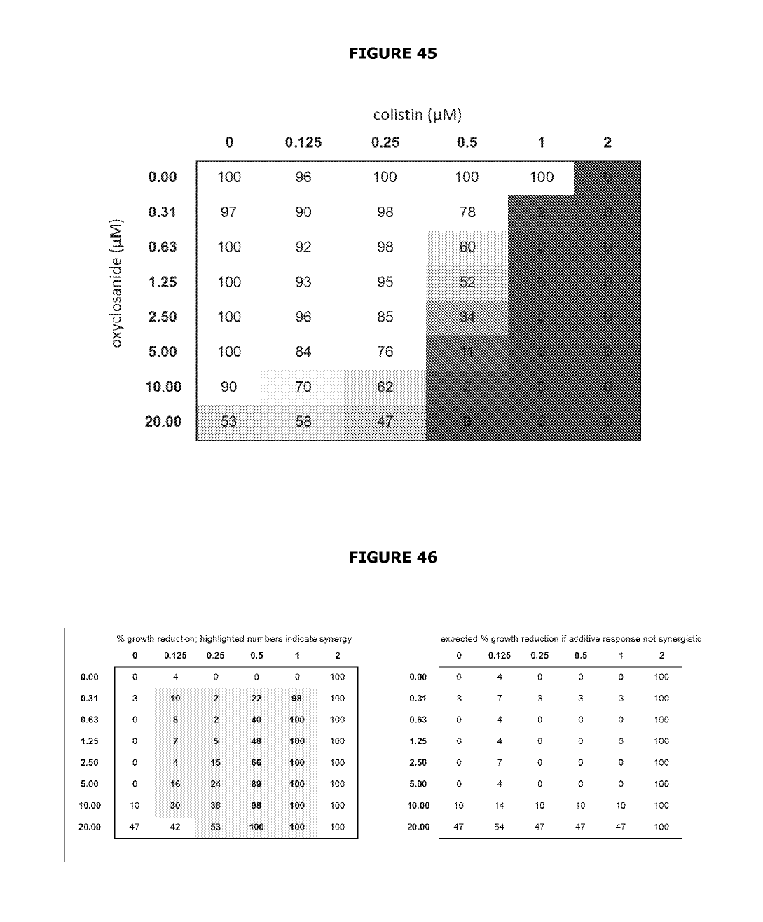

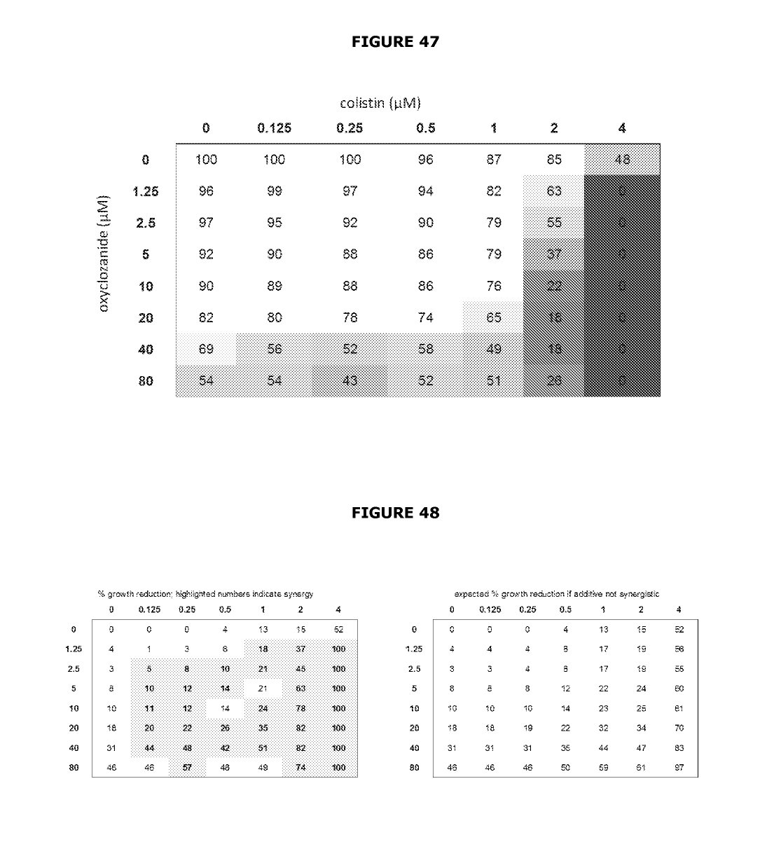

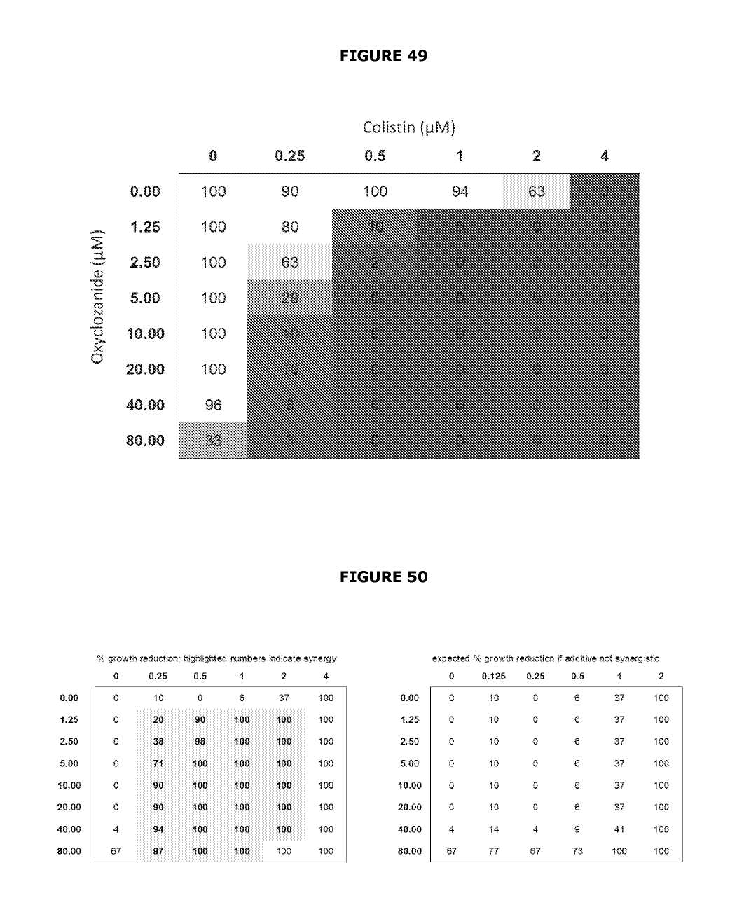

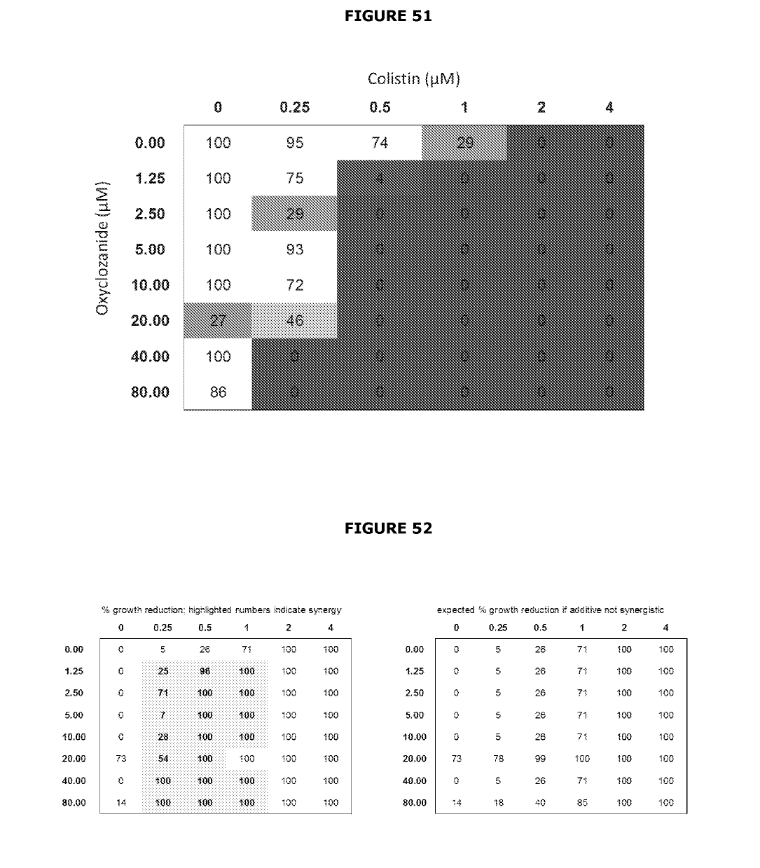

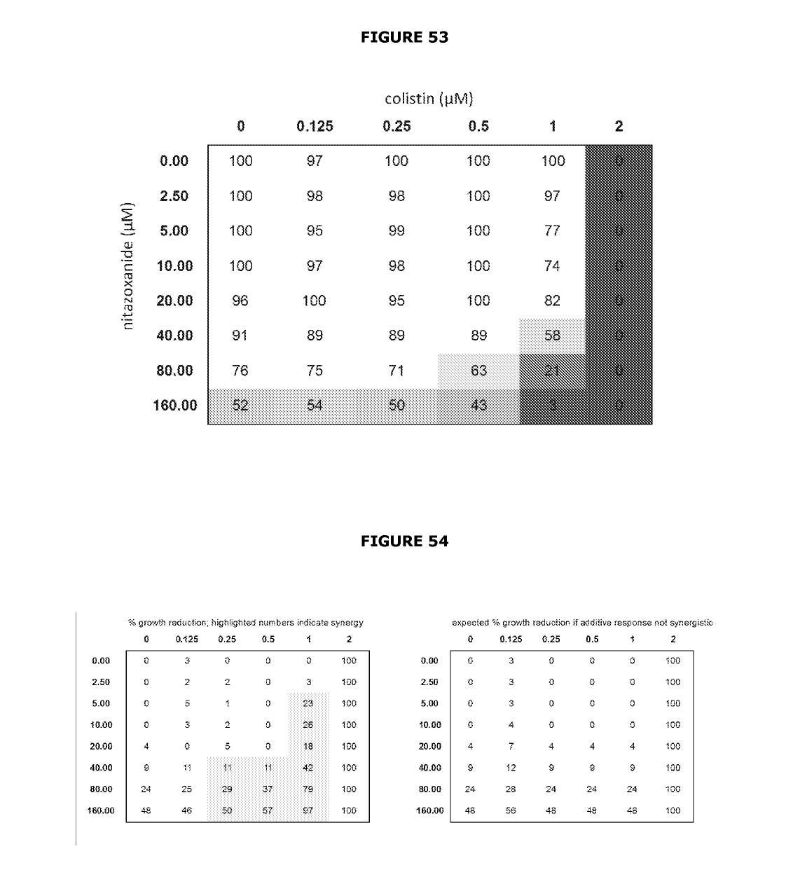

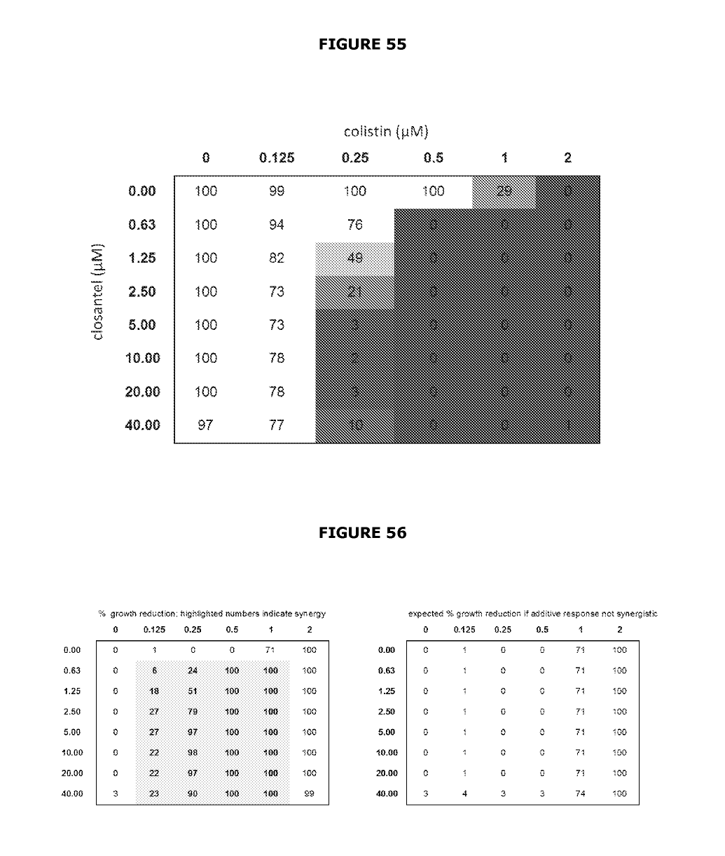

The effect of oxyclozanide (FIGS. 45-52), nitazoxanide (FIGS. 53 and 54), and closantel (FIGS. 55 and 56) further demonstrates that salicylamide compounds related to niclosamide exhibit similar synergistic effects when used in combination with various membrane permeabilizing antibiotics (e.g. colistin) against a wide range of Gram negative bacteria.

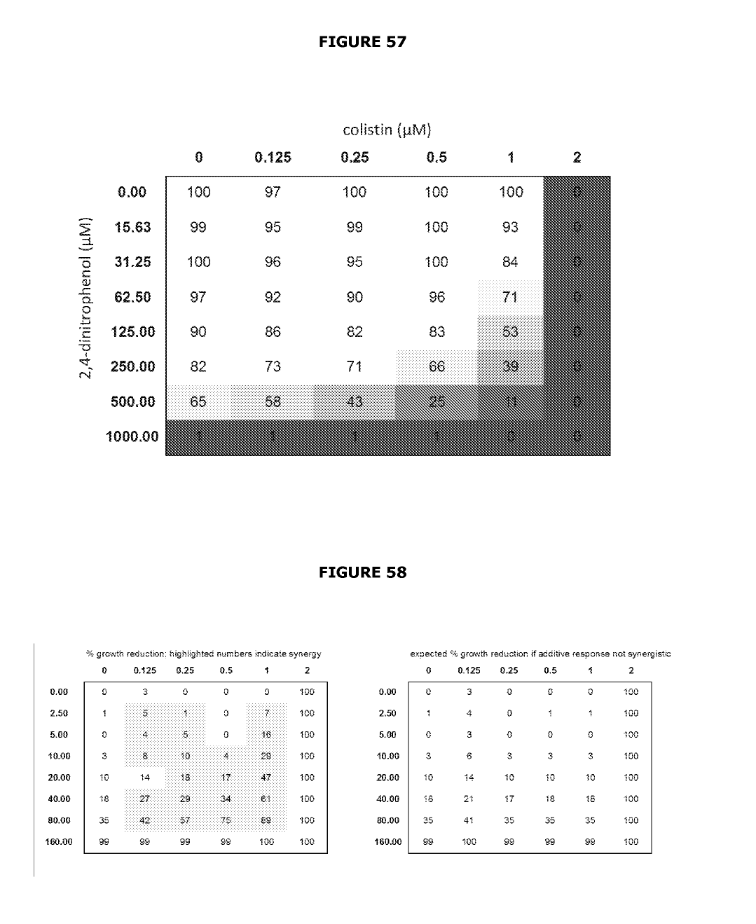

FIGS. 57 and 58 demonstrate that the combination of colistin and a membrane-uncoupling agent, namely, 2,4-dinitrophenol, exhibited only weak synergistic effect and does not achieve the same level of growth inhibition against E. coli, at similar concentration when compared to the combination of colistin and niclosamide (e.g. FIGS. 1, 2, 5, 6, 9, 10), or colistin and oxyclozanide (FIGS. 45, 46), or colistin and nitazoxanide (FIGS. 53, 54), or colistin and closantel (FIGS. 55, 56).

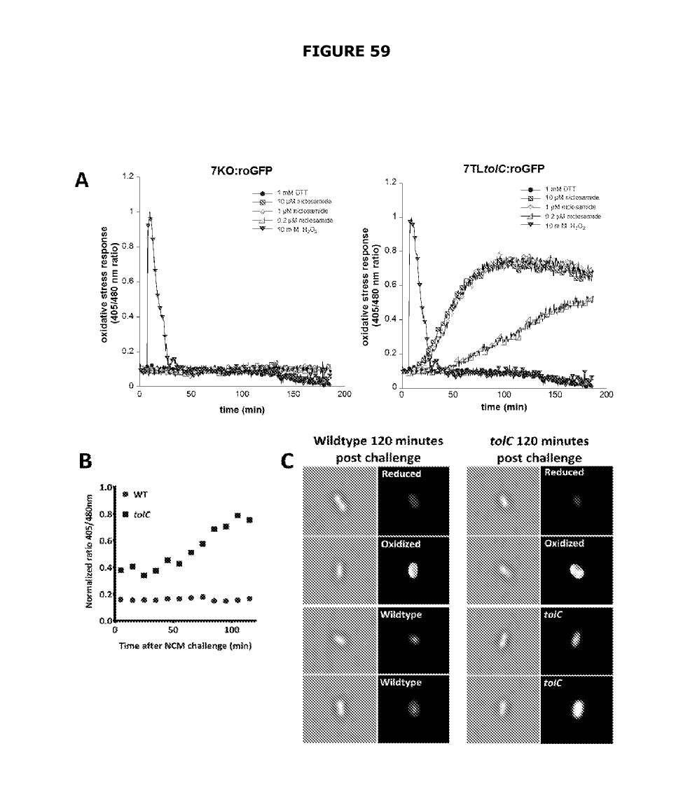

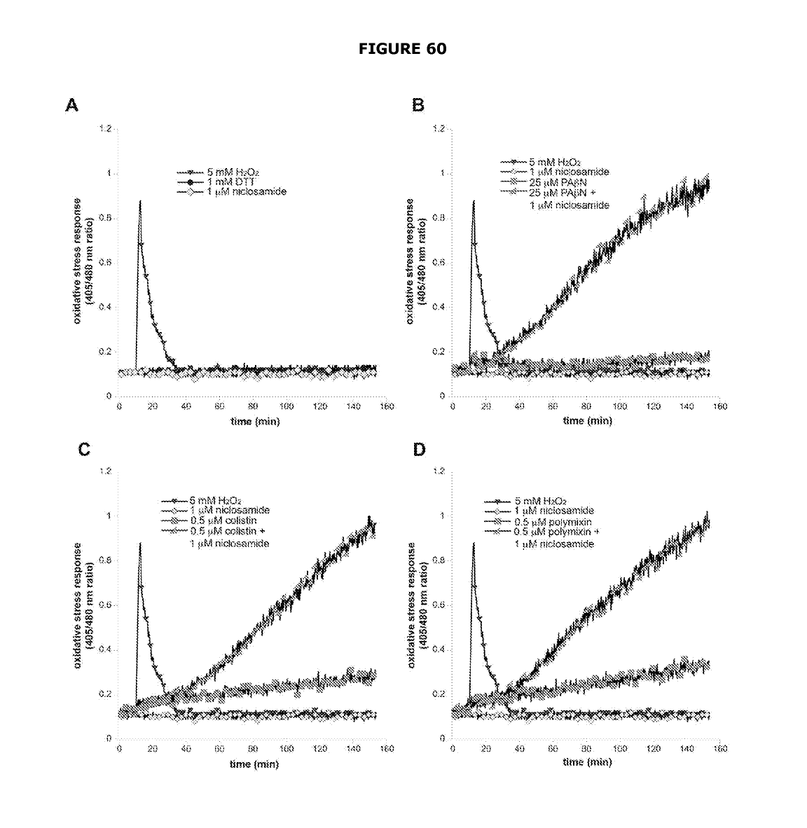

Without wishing to be bound by theory, Applicants hypothesize that the agent that increases the permeability of the bacterial cell membrane acts through a mechanism involving redox stress. Refer to, for example, Example 2 and FIGS. 59 and 60. Specifically, niclosamide causes an irreversible increase in intracellular redox stress when administered in combination with a membrane permeabilizing agent (e.g., polymyxin B, or colistin), or an agent that inhibits the TolC efflux pump of Gram negative bacteria (e.g., PAβN), or when administered in a strain with impaired efflux mechanisms (e.g., E. coli strain 7KOΔtolC, which harbors an in-frame deletion of the native tolC gene).

Collectively, these data provide direct and unambiguous evidence to demonstrate the synergistic effects of the various antibiotic compositions described herein, and their specific activity against wide range of Gram negative bacteria.

Accordingly, in one aspect the present invention provides a pharmaceutical composition comprising a salicylamide compound and an agent that increases the permeability of a bacterial cell membrane.

In a related example, the salicylamide compound is any compound defined by:

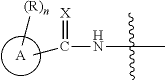

where A is an aryl or heteroaryl ring, e.g. a phenyl ring, (R)ₙ indicates that the aryl or heteroaryl ring may optionally be substituted with one or more substituents, and X is oxygen or another heteroatom such as sulfur. The group —C(=x)—NH— can be linked to ring A via the carbon or the nitrogen atom.

In a further related example, the salicylamide compound is a salicylanilide.

In yet a further related example, the salicylamide compound is selected from the group consisting of niclosamide, oxyclozanide, nitazoxanide and closantel.

In another related example, the salicylamide is niclosamide, or a niclosamide analogue defined by a compound of Formula I:

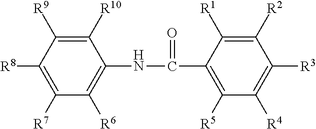

where R₁, R₂, R₃, R₄, R₅, R₆, R₇, R₈, R₉, and R₁₀ are as defined in Table 1. Refer below.

In another example, the agent that increases the permeability of the bacterial cell membrane is selected from the group consisting of hyperosmotic solutions, calcium ion chelators, surfactants, cationic or anionic peptides that disrupt cell membrane homeostasis and/or polarity, and non/receptor mediated permeabilizing agents including drug based agents that increase permeability of a bacterial cell membrane, as well as combinations thereof.

In a related example, the agent that increases the permeability of the bacterial cell membrane is a polymyxin, including but not limited to, polymyxin B and polymyxin E, as well as structural and/or functional analogues thereof. An example of a polymyxin E according to the present invention is colistin. Both terms are used interchangeably in the art, although colistin is in fact a mixture of polymyxin E1 and polymyxin E2.

In a further related example, agent that increases the permeability of the bacterial cell membrane may include cationic or anionic peptides that disrupt cell membrane homeostasis and/or polarity. An example of a cationic peptide according to this invention is gramicidin. Refer to (e.g.) Hurdle et al. (2011) Nat. Rev. Microbiol. 9(1):62-75 and Guilhelemelli et al. (2013) Front Microbiol. 4:353, which reviews are incorporated herein by reference.

The pharmaceutical composition according to the present invention may contain a pharmaceutically acceptable excipient or carrier. Further, the salicylamide compound may be formulated as a pharmaceutically acceptable salt or prodrug.

In another aspect the present invention provides a pharmaceutical composition comprising niclosamide and a polymyxin, wherein the polymyxin increases the permeability of a bacterial cell membrane.

In yet another aspect the present invention provides a pharmaceutical composition comprising a compound of Formula I and a polymyxin, including polymyxin B or polymyxin E, wherein the polymyxin increases the permeability of a bacterial cell membrane, and wherein the compound of Formula I is as defined:

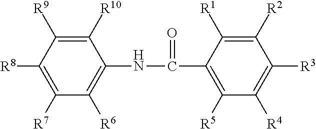

where R₁, R₂, R₃, R₄, R₅, R₆, R₇, R₈, R₉, and R₁₀ are as defined in Table 1.

In yet another aspect the present invention provides a pharmaceutical composition comprising oxyclozanide and a polymyxin, including polymyxin B or polymyxin E, wherein the polymyxin increases the permeability of a bacterial cell membrane.

In yet another aspect the present invention provides a pharmaceutical composition comprising nitazoxanide and a polymyxin, including polymyxin B or polymyxin E, wherein the polymyxin increases the permeability of a bacterial cell membrane.

In yet another aspect the present invention provides a pharmaceutical composition comprising closantel and a polymyxin, including polymyxin B or polymyxin E, wherein the polymyxin increases the permeability of a bacterial cell membrane.

In another aspect the present invention provides a pharmaceutical composition comprising niclosamide and gramicidin, wherein the gramicidin increases the permeability of a bacterial cell membrane.

In yet another aspect the present invention provides a pharmaceutical composition comprising a compound of Formula I and gramicidin, wherein the gramicidin increases the permeability of a bacterial cell membrane, and wherein the compound of Formula I is as defined:

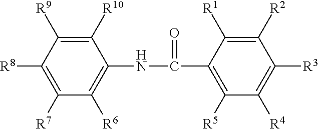

where R₁, R₂, R₃, R₄, R₅, R₆, R₇, R₈, R₉, and R₁₀ are as defined in Table 1.

In yet another aspect the present invention provides a pharmaceutical composition comprising oxyclozanide and gramicidin, wherein the gramicidin increases the permeability of a bacterial cell membrane.

In yet another aspect the present invention provides a pharmaceutical composition comprising nitazoxanide and gramicidin, wherein the gramicidin increases the permeability of a bacterial cell membrane.

In yet another aspect the present invention provides a pharmaceutical composition comprising closantel and gramicidin, wherein the gramicidin increases the permeability of a bacterial cell membrane.

In a further aspect the present invention provides a combination product comprising a salicylamide compound and an agent that increases the permeability of a bacterial cell membrane.

In yet a further aspect the present invention provides a synergistic combination comprising a salicylamide compound and an agent that increases the permeability of a bacterial cell membrane.

In another aspect the present invention provides an anti-bacterial agent comprising a salicylamide compound and an agent that increases the permeability of a bacterial cell membrane.

In yet another aspect the present invention provides a composition, including a biological composition, comprising a salicylamide compound and an agent that increases the permeability of a bacterial cell membrane.

In yet a further aspect the present invention provides a combination product, a synergistic combination, an anti-bacterial agent or a composition, including a biological composition, comprising niclosamide and a polymyxin, including polymyxin B or polymyxin E, wherein the polymyxin increases the permeability of a bacterial cell membrane.

In yet another aspect the present invention provides a combination product, a synergistic combination, an anti-bacterial agent or a composition, including a biological composition, comprising a compound of Formula I and a polymyxin, including polymyxin B or polymyxin E, wherein the polymyxin increases the permeability of a bacterial cell membrane, and wherein the compound of Formula I is as defined:

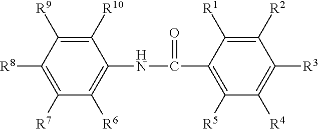

where R₁, R₂, R₃, R₄, R₅, R₆, R₇, R₈, R₉, and R₁₀ are as defined in Table 1.

In yet another aspect the present invention provides a combination product, a synergistic combination, an anti-bacterial agent or a composition, including a biological composition, comprising oxyclozanide and a polymyxin, including polymyxin B or polymyxin E, wherein the polymyxin increases the permeability of a bacterial cell membrane.

In yet another aspect the present invention provides a combination product, a synergistic combination, an anti-bacterial agent or a composition, including a biological composition, comprising nitazoxanide and a polymyxin, including polymyxin B or polymyxin E, wherein the polymyxin increases the permeability of a bacterial cell membrane.

In yet another aspect the present invention provides a combination product, a synergistic combination, an anti-bacterial agent or a composition, including a biological composition, comprising closantel and a polymyxin, including polymyxin B or polymyxin E, wherein the polymyxin increases the permeability of a bacterial cell membrane.

In another aspect the present invention provides a combination product, a synergistic combination, an anti-bacterial agent or a composition, including a biological composition, comprising niclosamide and gramicidin, wherein the gramicidin increases the permeability of a bacterial cell membrane.

In yet another aspect the present invention provides a combination product, a synergistic combination, an anti-bacterial agent or a composition, including a biological composition, comprising a compound of Formula I and gramicidin, wherein the gramicidin increases the permeability of a bacterial cell membrane, and wherein the compound of Formula I is as defined:

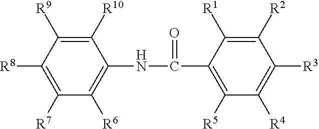

where R₁, R₂, R₃, R₄, R₅, R₆, R₇, R₈, R₉, and R₁₀ are as defined in Table 1.

In yet another aspect the present invention provides a combination product, a synergistic combination, an anti-bacterial agent or a composition, including a biological composition, comprising oxyclozanide and gramicidin, wherein the gramicidin increases the permeability of a bacterial cell membrane.

In yet another aspect the present invention provides a combination product, a synergistic combination, an anti-bacterial agent or a composition, including a biological composition, comprising nitazoxanide and gramicidin, wherein the gramicidin increases the permeability of a bacterial cell membrane.

In yet another aspect the present invention provides a combination product, a synergistic combination, an anti-bacterial agent or a composition, including a biological composition, comprising closantel and gramicidin, wherein the gramicidin increases the permeability of a bacterial cell membrane.

The combinations and compositions of the present invention, and as described herein, are particularly useful for the treatment or prevention of infection, particularly in humans, and for the prevention, reduction or elimination of biofilm formation, among other applications.

Accordingly, in yet a further aspect the present invention provides method for treating or preventing a bacterial infection in a patient or for reducing or eliminating formation of a bacterial biofilm comprising administering an antibiotically effective amount of a salicylamide compound and an agent that increases the permeability of a bacterial cell membrane, wherein the infection or biofilm comprises one or more Gram negative bacteria.

In a related example, the salicylamide compound and the agent that increases the permeability of a bacterial cell membrane is as defined above.

In another example, the Gram negative bacteria includes, but is not limited to Escherichia coli, including Escherichia coli strain MG1655; Enterobacter species, including but not limited to Enterobacter cloacae subsp. cloacae ATCC 13047; Salmonella enterica, including Salmonella enterica Serovar Typhimurium (SL1344); Pseudomonas species, including but not limited to Pseudomonas aeruginosa PAO1 and Pseudomonas syringae pv. actinidae, Klebsiella pneumoniae, including Klebsiella pneumoniae ATCC BAA-1705 and Acinetobacter baumannii, including Acinetobacter baumannii ATCC type strain 19606. In other examples, the bacteria are selected from the group consisting of Gram negative bacteria belonging to the genus of Shigella, Neisseria, Morexella, Legionella, Serratia, Haemophilus, Yersinia, Bordetella, Brucella, Campylobacter, Francisella, Helicobacter, Pasteurella, Vibrio and other Klebsiella and Salmonella. The genus of Shigella includes, but is not limited to, Shigella dysenteriae, Shigella flexneri, Shigella boydii, Shigella sonnei. The genus of Neisseria includes, but is not limited to, Neisseria gonorrhoeae and Neisseria meningitidis. The genus of Moraxella includes, but is not limited to, Moraxella catarrhalis, Moraxella lacunata and Moraxella bovis. The genus of Legionella includes, but is not limited to, Legionella pneumophila. The genus of Serratia includes, but is not limited to, Serratia marcescens, Serratia plymuthica, Serratia liquefaciens, Serratia rubidaea and Serratia odoriferae. The genus of Haemophilus includes, but is not limited to, Haemophilus aegyptius, Haemophilus ducreyi, Haemophilus influenzae, Haemophilus haemolyticus, Haemophilus parainfluenzae and Haemophilus parahaemolyticus. The genus of Yersinia includes, but is not limited to, Yersinia pestis and Yersinia pseudotuberculosis. The genus of Bordetella includes, but is not limited to, Bordetella bronchiseptica, Bordetella pertussis and Bordetella parapertussis. The genus of Brucella includes, but is not limited to, Brucella melitensis and Brucella abortus. The genus of Campylobacter includes, but is not limited to, Campylobacter jejuni and Campylobacter coli. The genus of Francisella includes, but is not limited to, Francisella tularensis and Francisella novicida. The genus of Helicobacter includes, but is not limited to, Helicobacter pylori. The genus of Pasteurella includes, but is not limited to, Pasteurella multocida and Pasteurella haemolytica. The genus of Vibrio includes, but is not limited to, Vibrio cholera, Vibrio vulnificus, Vibrio fischeri and Vibrio parahaemolyticus. In addition to Klebsiella pneumoniae, the genus Klebsiella includes, but is not limited to, Klebsiella granulomatis, Klebsiella oxytoca, Klebsiella michiganensis and Klebsiella variicola. In addition to Salmonella enterica, the genus Salmonella includes, but is not limited to, Salmonella bongori.

The compositions and methods according to the present invention may have utility in animal (e.g. mastitis for dairy cows), industry and infrastructure (e.g. biofilm prevention in water purification plants, food packaging etc) or agricultural applications.

As such, the present invention further provides a composition, synergistic combination or an anti-bacterial agent comprising a salicylamide compound and an agent that increases the permeability of a bacterial cell membrane for treating or preventing mastitis in an animal. The present invention also provides a method for treating or preventing mastitis in an animal comprising administering an antibiotically effective amount of a salicylamide compound and an agent that increases the permeability of a bacterial cell membrane, wherein the bacteria causing mastitis comprises one or more Gram negative bacteria.

In a related example, the animal is a cow.

In a further related example, the salicylamide compound is niclosamide. Advantageously, in addition to its surprising/unexpected activity against Gram negative bacteria in the presence of an agent that increases the permeability of a bacterial cell membrane, niclosamide, in isolation, also exhibits bactericidal activity against Gram positive bacteria. Accordingly, the composition, synergistic combination or an anti-bacterial agent according to the present invention are particularly useful in mastitis applications where a mix of both Gram negative (e.g. E. coli) and Gram positive (e.g. Staphylococcus aureus and Group D Streptococci including Streptococcus uberis) may cause infection of the cow udder/teats. In yet a further related example, the synergistic combination or an anti-bacterial agent for use in preventing or treating mastitis according to the present invention may be administered as a spray to the cow udder/teats.

In another aspect the present invention provides use of a combination of a salicylamide compound and an agent that increases the permeability of a bacterial cell membrane or a composition comprising a salicylamide compound and agent that increases the permeability of a bacterial cell membrane, as a medicament.

In another aspect the present invention provides a combination a salicylamide compound and an agent that increases the permeability of a bacterial cell membrane for use in the preparation of a pharmaceutical composition.

A biofilm has the potential to cause infection in a wound and/or burn or causes an infection on or in an in-dwelling medical device. Alternatively, formation of bacterial biofilms occurs within preparative machinery for the food industry, on packaging used by the food industry, within storage tanks used for water or other liquids, or within machinery at water treatment plants, all of which have the potential to increase the risk of infection arising from human or animal contact with consumable products. Further, the accumulation of bacteria via biofilm formation on surfaces such as hospital beds, bathrooms and doors connecting wards etc also has the ability to expose humans to risk on infection.

Accordingly, the ability to not only treat or prevent a bacterial infection in humans (and animals), but to reduce or eliminate formation of bacterial biofilms is an equally important consideration for use of the combination products and compositions of this invention.

In certain embodiments, the combination products or compositions according to the invention may further comprise one or more bactericidal or bacteriostatic agents. Examples of bactericidal agents include, but are not limited to, beta lactam antibiotics (e.g. penicillin derivatives, cephalosporins, monobactams, carbapenems), vancomycin, daptomycin, fluoroquinolones, metronidazole, nitrofurantoin, co-trimoxazole or telithromycin Examples of bacteriostatic agents include, but are not limited to tetracyclines, macrolides, sulfonamides, lincosamides, oxazolidinone, tigecycline, novobiocin, nitrofurantoin, spectinomycin, trimethoprim, chloramphenicol, ethambutol or clindamycin.

The rise in antibiotic resistance is having a profound impact on the healthcare industry, and the need to provide alternative medicines to combat bacterial infection (i.e. to treat or prevent infection) is growing increasingly important. Accordingly, in another aspect the present invention provides use of a salicylamide compound and an agent that increases the permeability of a bacterial cell membrane in the manufacture of a medicament or a combination of a salicylamide compound and an agent that increases the permeability of a bacterial cell membrane for use in the manufacture of a medicament.

In yet another aspect the present invention provides the use of a salicylamide compound and an agent that increases the permeability of a bacterial cell membrane in the manufacture of a medicament for treating or preventing a bacterial infection in a patient, wherein the bacteria causing infection comprise Gram negative bacteria.

In yet a further aspect the present invention provides an article of manufacture comprising package material containing a salicylamide compound and an agent that increases the permeability of a bacterial cell membrane together with instructions for use in treating or preventing a bacterial infection in a patient or for reducing or eliminating formation of a bacterial biofilm, wherein the infection or biofilm comprises one or more Gram negative bacteria.

In another aspect the present invention provides an article of manufacture comprising package material containing niclosamide and a polymyxin, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing a compound of Formula I and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria,

and wherein the compound of Formula I is as defined:

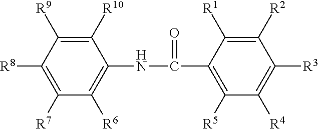

and R₁, R₂, R₃, R₄, R₅, R₆, R₇, R₈, R₉, and R₁₀ are as defined in Table 1.

In yet another aspect the present invention provides an article of manufacture comprising package material containing oxyclozanide and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing nitazoxanide and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing closantel and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In another aspect the present invention provides an article of manufacture comprising package material containing a niclosamide and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing a compound of Formula I and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria,

and wherein the compound of Formula I is as defined:

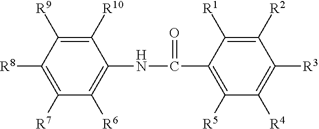

and R₁, R₂, R₃, R₄, R₅, R₆, R₇, R₈, R₉, and R₁₀ are as defined in Table 1.

In yet another aspect the present invention provides an article of manufacture comprising package material containing oxyclozanide and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing nitazoxanide and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

In yet another aspect the present invention provides an article of manufacture comprising package material containing closantel and gramicidin, wherein the gramicidin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria.

The present invention further contemplates pharmaceutical compositions, combination products, synergistic combinations, anti-bacterial agents, compositions, including biological compositions, and articles of manufacture which exclude niclosamide and colistin.

Accordingly, in another aspect the present invention provides a pharmaceutical composition comprising a salicylamide compound and an agent that increases the permeability of a bacterial cell membrane, provided that the pharmaceutical composition does not contain niclosamide and colistin.

In another aspect the present invention provides a pharmaceutical composition comprising niclosamide and a polymyxin, including polymyxin B or polymyxin E, wherein the polymyxin increases the permeability of a bacterial cell membrane, provided that the polymyxin is not colistin.

In yet another aspect the present invention provides a pharmaceutical composition comprising a compound of Formula I and a polymyxin, including polymyxin B or polymyxin E, wherein the polymyxin increases the permeability of a bacterial cell membrane, and wherein the compound of Formula I is as defined:

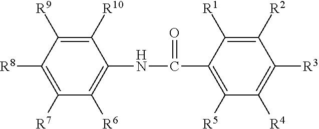

where R₁, R₂, R₃, R₄, R₅, R₆, R₇, R₈, R₉, and R₁₀ are as defined in Table 1, provided that the compound of Formula I is not niclosamide.

In a further aspect of the present invention there is provided a combination product comprising a salicylamide compound and an agent that increases the permeability of a bacterial cell membrane, provided that the combination product does not contain niclosamide and colistin.

In yet a further aspect of the present invention there is provided a synergistic combination comprising a salicylamide compound and an agent that increases the permeability of a bacterial cell membrane, provided that the synergistic combination does not contain niclosamide and colistin.

In yet a further aspect of the present invention there is provided an anti-bacterial agent comprising a salicylamide compound and an agent that increases the permeability of a bacterial cell membrane, provided that the anti-bacterial agent does not contain niclosamide and colistin.

In yet a further aspect the present invention provides a composition, including a biological composition, comprising a salicylamide compound and an agent that increases the permeability of a bacterial cell membrane, provided that the composition does not contain niclosamide and colistin.

In yet a further aspect the present invention provides a combination product, a synergistic combination, an anti-bacterial agent or a composition, including a biological composition, comprising niclosamide and a polymyxin, including polymyxin B or polymyxin E, wherein the polymyxin increases the permeability of a bacterial cell membrane, provided that the polymyxin is not colistin.

In yet another aspect the present invention provides a combination product, a synergistic combination, an anti-bacterial agent or a composition, including a biological composition, comprising a compound of Formula I and a polymyxin, including polymyxin B or polymyxin E, wherein the polymyxin increases the permeability of a bacterial cell membrane, and wherein the compound of Formula I is as defined:

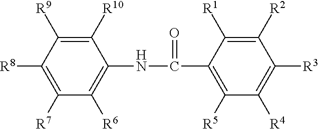

where R₁, R₂, R₃, R₄, R₅, R₆, R₇, R₈, R₉, and R₁₀ are as defined in Table 1, provided that the compound of Formula I is not niclosamide.

In yet another aspect the present invention provides a pharmaceutical composition as described herein or a combination product, a synergistic combination, an anti-bacterial agent or a composition as described herein for use in:
(i) treating or preventing a bacterial infection in a human or non-human animal; or
(ii) reducing or eliminating formation of a bacterial biofilm
wherein the infection or biofilm comprises one or more Gram negative bacteria.

In yet a further aspect the present invention provides an article of manufacture comprising package material containing a salicylamide compound and an agent that increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria, provided that the package material does not contain niclosamide and colistin.

In another aspect the present invention provides an article of manufacture comprising package material containing a niclosamide and a polymyxin, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria, provided that the polymyxin is not colistin.

In yet another aspect the present invention provides an article of manufacture comprising package material containing a compound of Formula I and a polymyxin, including polymyxin B or polymyxin E, wherein the polymyxin increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria,

and wherein the compound of Formula I is as defined:

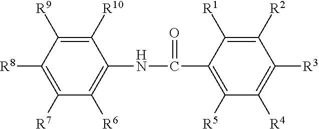

and R₁, R₂, R₃, R₄, R₅, R₆, R₇, R₈, R₉, and R₁₀ are as defined in Table 1, provided that the compound of Formula I is not niclosamide.

The present invention further contemplates methods for treating or preventing a bacterial infection in a patient or for reducing or eliminating formation of a bacterial biofilm by administering antibiotically effective amounts of niclosamide and colistin to the patient or to the biofilm, provided that the bacteria causing infection or biofilm formation is not Klebsiella pneumoniae and/or Acinetobacter baumannii.

Accordingly, in yet another aspect the present invention provides a method for treating or preventing a bacterial infection in a patient or for reducing or eliminating formation of a bacterial biofilm comprising administering to the patient or to the biofilm an antibiotically effective amount of a salicylamide compound and an agent that increases the permeability of a bacterial cell membrane, wherein the infection or biofilm comprises one or more Gram negative bacteria, and wherein when the salicylamide is niclosamide and the agent that increases the permeability of a bacterial cell membrane is colistin, the Gram negative bacteria is not Klebsiella pneumoniae and/or Acinetobacter baumannii.

In yet a further aspect the present invention provides method for treating or preventing a bacterial infection in a patient or for reducing or eliminating formation of a bacterial biofilm comprising administering an antibiotically effective amount of niclosamide and colistin to the patient or to the biofilm, provided that the bacteria causing infection or biofilm formation is not Klebsiella pneumoniae and/or Acinetobacter baumannii.

### Salicylamides

Those skilled in the art will understand that any suitable salicylamide compound having antibiotic activity may be used in the combinations and compositions of the invention, as well as derivatives or analogues thereof. In certain examples, the salicylamide compound exhibits antibiotic activity against Gram negative bacteria. Suitable salicylamide compounds for use in the present invention preferably include the structural moiety:

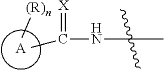

where A is an aryl or heteroaryl ring, e.g. a phenyl ring, (R)ₙ indicates that the aryl or heteroaryl ring may optionally be substituted with one or more substituents, and X is oxygen or another heteroatom such as sulfur. The group —C(=x)—NH— can be linked to ring A via the carbon or the nitrogen atom. Preferably the salicylamide compound includes one or more nitro groups.

In certain examples, the salicylamide compound is a salicylanilide compound that includes two or more aryl groups, e.g. two or more phenyl rings, each of which may optionally be substituted, for example as shown in formula (I) below. Alternatively, the salicylamide compound may include one or more heteroaryl groups. The salicylamide compound may include a heteroatom, such as sulfur, in place of the oxygen of the amide group. The term “salicylamide compound” is intended to include all such derivatives and analogues.

A preferred salicylamide compound is the salicylanilide compound niclosamide (N-(2′-chloro-4′-nitrophenyl)-5-chlorosalicylamide), the structure of which is shown below.

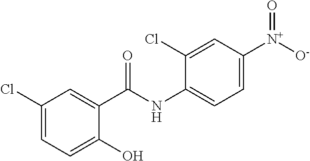 [SMILES: O=C(Nc1ccc([N+](=O)[O-])cc1Cl)c1cc(Cl)ccc1O]

Salt forms of niclosamide are known, including an ethanolamine salt and a piperizine salt. Furthermore, a monohydrate form of niclosamide is also known. Any suitable pharmaceutically acceptable excipient, including salts or hydrates, may be used in the compositions and combinations of the present invention.

Other examples of salicylamide compounds include analogues of niclosamide. Analogues of salicylamide compounds are known, for example those described in US2011/0183889, which is incorporated herein by reference. Suitable niclosamide analogues for use in the compositions and combinations of the present invention include, but are not limited to, those described by general Formula (I), wherein R¹-R¹⁰ are as defined herein, including those listed in Table 1 below. Other suitable niclosamide analogues for use in the present invention include approved drug analogues of niclosamide.

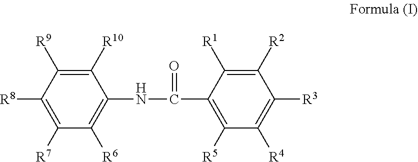

TABLE 1

| Compound | Substituents | | | | | | | | | |
|---|---|---|---|---|---|---|---|---|---|---|
| Number | R¹ | R² | R³ | R⁴ | R⁵ | R⁶ | R⁷ | R⁸ | R⁹ | R¹⁰ |
| 1 | OH | H | Cl | H | H | Cl | H | NO₂ | H | H |
| 2 | OH | Cl | H | H | H | Cl | H | NO₂ | H | H |
| 3 | OH | H | H | H | Cl | Cl | H | NO₂ | H | H |
| 4 | OH | H | H | Cl | H | H | Cl | NO₂ | H | H |
| 5 | OH | Cl | H | H | H | H | Cl | NO₂ | H | H |
| 6 | OH | H | Cl | H | H | H | Cl | NO₂ | H | H |
| 7 | OH | H | H | H | Cl | H | Cl | NO₂ | H | H |
| 8 | OH | H | H | Cl | H | Cl | NO₂ | H | H | H |
| 9 | OH | H | H | Cl | H | Cl | H | H | H | NO₂ |
| 10 | OH | H | H | H | Cl | Cl | H | H | NO₂ | H |
| 11 | H | OH | H | Cl | H | Cl | H | NO₂ | H | H |
| 12 | H | H | OH | Cl | H | Cl | H | NO₂ | H | H |
| 13 | Cl | OH | H | H | H | Cl | H | NO₂ | H | H |
| 14 | H | OH | Cl | H | H | Cl | H | NO₂ | H | H |
| 15 | H | OH | H | H | Cl | Cl | H | NO₂ | H | H |
| 16 | H | H | OH | H | Cl | Cl | H | NO₂ | H | H |
| 17 | OH | H | Cl | H | H | Cl | NO₂ | H | H | H |
| 18 | OH | Cl | H | H | H | Cl | NO₂ | H | H | H |
| 19 | OH | H | H | H | Cl | Cl | NO₂ | H | H | H |
| 20 | OH | H | H | Cl | H | F | H | NO₂ | H | H |
| 21 | OH | H | H | F | H | Cl | H | NO₂ | H | H |
| 22 | OH | H | H | Cl | H | Br | H | NO₂ | H | H |
| 23 | OH | H | H | Br | H | Cl | H | NO₂ | H | H |
| 24 | OH | H | H | Br | H | F | H | NO₂ | H | H |
| 25 | OH | H | H | F | H | Br | H | NO₂ | H | H |
| 26 | OH | H | H | Br | H | Br | H | NO₂ | H | H |
| 27 | OH | H | H | F | H | F | H | NO₂ | H | H |
| 28 | OH | H | H | Cl | H | Cl | H | NO₂ | H | H |
| 29 | OH | H | Cl | H | H | Br | H | H | H | H |
| 30 | H | H | OH | Cl | H | Br | H | NO₂ | H | H |
| 31 | OH | Cl | H | H | H | Br | H | NO₂ | H | H |
| 32 | OH | H | Cl | H | H | Cl | H | H | H | NO₂ |
| 33 | H | H | OH | Cl | H | Cl | H | H | H | NO₂ |
| 34 | OH | Cl | H | H | H | Cl | H | H | H | NO₂ |
| 35 | H | H | OH | F | H | Cl | H | H | H | NO₂ |
| 36 | H | H | OH | Br | H | Cl | H | H | H | NO₂ |
| 37 | H | OH | H | Cl | H | Cl | H | H | H | NO₂ |
| 38 | OH | H | Cl | H | H | F | H | NO₂ | H | H |
| 39 | H | H | OH | Cl | H | F | H | NO₂ | H | H |
| 40 | OH | Cl | H | H | H | F | H | NO₂ | H | H |
| 41 | H | OH | H | Cl | H | F | H | NO₂ | H | H |
| 42 | OH | H | H | H | Cl | Br | H | NO₂ | H | H |
| 43 | OH | H | H | H | Cl | F | H | NO₂ | H | H |
| 44 | OH | H | H | H | Cl | Cl | H | H | NO₂ | H |
| 45 | Cl | OH | H | H | H | Br | H | NO₂ | H | H |
| 46 | Cl | OH | H | H | H | Cl | NO₂ | H | H | H |
| 47 | Cl | OH | H | H | H | F | H | NO₂ | H | H |
| 48 | Cl | OH | H | H | H | H | Cl | NO₂ | H | H |
| 49 | Cl | OH | H | H | H | Cl | H | H | NO₂ | H |
| 50 | H | OH | Cl | H | H | Br | H | NO₂ | H | H |
| 51 | H | OH | Cl | H | H | Cl | NO₂ | H | H | H |
| 52 | H | OH | Cl | H | H | F | H | NO₂ | H | H |
| 53 | H | OH | Cl | H | H | H | Cl | NO₂ | H | H |
| 54 | H | OH | Cl | H | H | Cl | H | H | NO₂ | H |
| 55 | H | OH | H | H | Cl | Br | H | NO₂ | H | H |
| 56 | H | OH | H | H | Cl | Cl | NO₂ | H | H | H |
| 57 | H | OH | H | H | Cl | F | H | NO₂ | H | H |
| 58 | H | OH | H | H | Cl | H | Cl | NO₂ | H | H |
| 59 | H | OH | H | H | Cl | Cl | H | H | NO₂ | H |
| 60 | H | H | OH | H | Cl | Cl | NO2 | H | H | H |
| 61 | H | H | OH | H | Cl | F | H | NO₂ | H | H |
| 62 | H | H | OH | H | Cl | H | Cl | NO₂ | H | H |
| 63 | H | H | OH | H | Cl | Cl | H | H | NO₂ | H |
| 64 | OH | H | Cl | H | H | Cl | H | H | NO₂ | H |
| 65 | H | H | OH | Cl | H | Cl | H | H | NO₂ | H |
| 66 | OH | Cl | H | H | H | Cl | H | H | NO₂ | H |
| 67 | OH | H | H | Br | H | Cl | H | H | NO₂ | H |
| 68 | OH | H | H | F | H | Cl | H | H | NO₂ | H |
| 69 | H | OH | H | Cl | H | Cl | H | H | NO₂ | H |

Other salicylamide compounds that are suitable for use in the combinations and compositions of the present invention include, but are not limited to, oxyclozanide (2,3,5-trichloro-N-(3,5-dichloro-2-hydroxyphenyl)-6-hydroxybenzamide), closantel (N-[5-Chloro-4-[(4-chlorophenyl)-cyanomethyl]-2-methylphenyl]-2-hydroxy-3,5-diiodobenzamide), rafoxanide (N-[3-chloro-4-(4-chlorophenoxy)phenyl]-2-hydroxy-3,5-diiodobenzamide), flusalan (3,5-dibromo-2-hydroxy-N-[3-(trifluoromethyl)phenyl]benzamide), tribromsalan (3,5-dibromo-N-(4-bromophenyl)-2-hydroxybenzamide), dibromsalan (5-Bromo-N-(4-bromophenyl)-2-hydroxybenzamide), resorantel (N-(4-bromophenyl)-2,6-dihydroxybenzamide), clioxanide (acetic acid 2-(4-chloro-phenylcarbamoyl)-4,6-diiodo-phenyl ester), 4′-chloro-5-nitrosalicylanilide, 2′-chloro-5′-methoxy-3-nitrosalicylanilide, 2′-methoxy-3,4′-dinitrosalicylanilide, 2′,4′-dimethyl-3-nitrosalicylanilide, 4′,5′-dibromo-3-nitrosalicylanilide, 2′-chloro-3,4′-dinitrosalicylanilide, 2′-ethyl-3-nitrosalicylanilide, 2′-bromo-3-nitrosalicylanilide.

The structure of oxyclozanide is shown below:

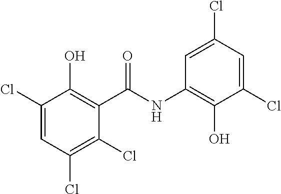 [SMILES: O=C(Nc1cc(Cl)cc(Cl)c1O)c1c(O)c(Cl)cc(Cl)c1Cl]

The structure of closantel is shown below:

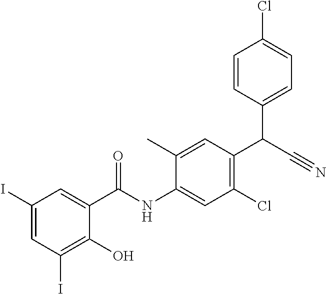 [SMILES: Cc1cc(C(C#N)c2ccc(Cl)cc2)c(Cl)cc1NC(=O)c1cc(I)cc(I)c1O]

The present invention also includes other salicylamide compounds, such as those containing one or more heteroaryl rings. The heteroaryl ring(s) may have one or more substituents. One example of such compounds is nitazoxanide (2-acetyloxy-N-(5-nitro 2-thiazolyl)benzamide), shown below.

Nitazoxanide

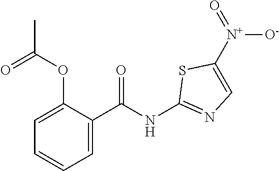 [SMILES: CC(=O)Oc1ccccc1C(=O)Nc1ncc([N+](=O)[O-])s1]

The invention furthermore includes other salicylamide compounds, such as those where the oxygen of the amide group is replaced by another heteroatom. One example of such compounds is brotianide (3,4′-dibromo-5-chlorothiosalicylanilide) shown below.

Brotianide

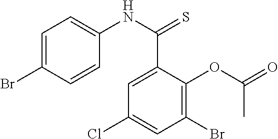 [SMILES: CC(=O)Oc1c(Br)cc(Cl)cc1C(=S)Nc1ccc(Br)cc1]

Some of the above-mentioned salicylamide compounds are commercially available. Others can readily be prepared by methods known to those skilled in the art. For example, WO 2004/006906, which is incorporated herein by reference, describes methods for preparing niclosamide analogues.

Agents that Increase Permeability of Bacterial Cell Membranes

The cells of prokaryotic organisms, including bacteria, are surrounded by a cell membrane composed primarily of two layers of phospholipids. When intact, this membrane prevents certain classes of chemical compounds from entering the cell. Compounds which can readily cross the membrane and enter or leave the cell are said to be membrane permeant. Membrane impermeant compounds are those which are excluded by the intact membrane when outside the cell, and/or which are retained by the membrane when formed inside the cell by intrinsic metabolic activity or by intracellular transformation of administered permeant compounds into impermeant compounds.

Although there are differences among cells in permeability to some compounds, it is widely believed that certain classes of compounds, including organic compounds bearing at least two positive charges and most negatively charged organic compounds, are impermeant to the cells of bacteria, protists, fungi, plants, and animals.

In bacterial and eukaryotic cells with intact membranes, there is typically a difference of electrical potential across the cell membrane, with the interior negative by between 5 and 200 mV with respect to the exterior. This membrane potential is generated by differences in concentrations of inorganic ions, to which membrane permeability is restricted, inside and outside the membrane. Membrane potential will be reduced to zero if the membrane develops holes large enough to permit inorganic ions to cross freely, as may occur when cells are killed by heating or by freezing and thawing; under these circumstances, the membrane typically becomes permeable to dyes such as propidium iodide. Several classes of chemical compounds can also alter membrane potential; these include ionophores, which carry inorganic ions through the membrane or form channels in the membrane allowing inorganic ions to pass through readily.

Surprisingly, Applicants have discovered that the specific combination of drug-based agents which increase the permeability of bacterial cell membranes can potentiate the effect of certain antibiotic classes, including (e.g.) salicylamides, which when combined demonstrate unexpected bactericidal activity against Gram negative bacteria.

Those skilled in the art will understand that an agent that increases the permeability of bacterial cell membrane used in the compositions and combinations of the present invention includes any agent sufficient to disrupt the integrity of the cell membrane. Through increased permeability of the cell membrane, Gram negative bacteria become surprisingly more susceptible to antibiotics including, for example, the salicylamide compounds described herein.

The term “increased permeability” as used herein, is defined as having a property of allowing an increased amount of a drug, such as an antibiotic, to travel through a cellular barrier relative to a cellular barrier that has not been exposed to the agent. The cellular barrier refers to a cellular structure such as a membrane junction and/or a cell membrane that acts to inhibit drug movement into or between cells that would otherwise occur through, for example, active or passive diffusion. A membrane junction refers to a junction between cell membranes of adjacent cells such as tight junctions, desmosomes and gap junctions. Obviously, cell membrane refers to the plasma membrane that encloses a cell's contents such as the cytoplasm and nucleus. For example, the amount of drug/antibiotic taken up by bacteria that has been exposed to an agent that increases the permeability of its cell membrane could be two to twenty times greater than a bacteria cell that has not been exposed to the agent.

An agent that increases the permeability of the cell membrane must be applied at a concentration sufficient to increase the permeability of the bacterial cell membrane, which then makes it susceptible or more susceptible to a particular drug such as an antibiotic or antibiotic combination. The application of the agent that increases the permeability of the cell membrane is concomitantly or subsequently followed by the administration of a drug to allow the drug to penetrate into the cytoplasm and take effect on its cellular target. Many different types of compounds may be used as agents to increase the permeability of cell membranes, and various devices may be used to deliver these agents and/or the drug. Moreover, the invention has many different and potential clinical applications.

The amount of drug administered in conjunction with, or after administration of, the agent that increases the permeability of the cell membrane may also be determined on an individual basis and is based, at least in part, on consideration of the individual's size, the specific disease, the severity of the symptoms to be treated, the result sought, and other factors. Standard pharmacokinetic test procedures employing laboratory animals to determine dosages are understood by one of ordinary skill in the art.

Examples of agents that increase the permeability of bacterial cell membranes used in the compositions, combinations and methods according to the present invention include, but are not limited to, hyperosmotic solutions, calcium ion chelators, surfactants, cationic or anionic peptides (including gramicidin) that disrupt cell membrane homeostasis and/or polarity and receptor mediated permeabilizing agents including drug based agents that increase permeability of a bacterial cell membrane, as well as combinations thereof.

Examples of calcium ion chelators useful for this purpose include iminodiacetic acid (IDA), nitriloacetic acid (NTA), ethylenediaminomonoacetic acid (EDMA), ethylenediaminodiacetic acid (EDDA), and ethylenediaminotetraacetic acid (EDTA). Extensive literature is available concerning the use of EDTA, because it is used as an excipient in many drug compositions. In one example, the concentration of calcium ion chelator required to decrease intracellular calcium ion concentrations can be from about 0.01 mM to 1 M, for example about 1 mM.

Examples of useful ionic surfactants include, but are not limited to, sodium taurodihydrofusidate, sodium salicylate, sodium caprate, and sodium glycocholate. Examples of useful non-ionic surfactants include, but are not limited to cholylsarcosine, isopropyl myristate, partially hydrolyzed triglycerides, fatty-acid sugar derivatives, and oleic acid derivatives. These surfactants may be administered in concentrations ranging from 0.0001% to 10%, more narrowly about 0.001 to 1%, by example about 0.1%. Although ionic surfactants tend to be slightly more effective in fluidizing the membrane, they also tend to be slightly more irritating.

Examples of cationic or anionic peptides that disrupt cell membrane homeostasis and/or polarity are (e.g.) described in Hurdle et al. (2011) and Guihelemelli et al. (2013) ibid and includes gramicidin.

Examples of drug based agents that increase permeability of a bacterial cell membrane useful in the compositions, combinations and methods according to the present invention, include, but are not limited to polymyxins, including polymyxin B and polymyxin E (e.g. colistin).

Specifically, there are two polymyxins available for use in the clinic, namely polymyxin B and polymyxin E (or colistin); they consist of a cyclic peptide with a long hydrophobic tail. Both colistin and polymyxin B were discovered in the 1940s and have been used extensively to combat Gram negative infections. However, owing to nephrotoxic and neurotoxic side-effects their use waned throughout the 1970s (Velkov et al. (2013) Future Microbiol. 8(6):711-24). More recently, their use as an antibiotic of last resort has increased, owing to the extensive spread of bacteria resistant to front-line antibiotics (e.g. Li et al. (2006) Lancet Infect. Dis. 6(9):589-601). For many multidrug resistant Enterobacteriaceae (especially K. pneumoniae and Escherichia coli) polymyxin antibiotics are the only remaining therapeutic option (Velkov et al. (2013) ibid).

Structural/functional analogues of polymyxin B, which differ in the N-terminal fatty acyl group and amino acid residue at position-6 and position-7, include, but are not limited to, polymyxin B₁ [fatty acyl=(S)-6-methyloctanoyl; Pos-6=D-Phe; Pos-7=Leu], polymyxin B₁-Ile [fatty acyl=(S)-6-methyloctanoyl; Pos-6=D-Phe; Pos-7=Ile], polymyxin B₂ [fatty acyl=6-methylheptanoyl; Pos-6=D-Phe; Pos-7=Leu], polymyxin B₃ [fatty acyl=octanoyl; Pos-6=D-Phe; Pos-7=Leu], polymyxin B₄ [fatty acyl=heptanoyl; Pos-6=D-Phe; Pos-7=Leu], polymyxin B₅ [fatty acyl=nonanoyl; Pos-6=D-Phe; Pos-7=Leu] and polymyxin B₆ [fatty acyl=3-hydroxy-6-methyloctanoyl; Pos-6=D-Phe; Pos-7=Leu].

Structural/functional analogues of polymyxin E, which differ in the N-terminal fatty acyl group and amino acid residue at position-6 and position-7, include, but are not limited to, polymyxin E₁ [fatty acyl=(S)-6-methyloctanoyl; Pos-6=D-Leu; Pos-7=Leu], polymyxin E₂ [fatty acyl=6-methylheptanoyl; Pos-6=D-Leu; Pos-7=Leu], polymyxin E₃ [fatty acyl=octanoyl; Pos-6=D-Leu; Pos-7=Leu], polymyxin E₄ [fatty acyl=heptanoyl; Pos-6=D-Leu; Pos-7=Leu], polymyxin E₇ [fatty acyl=7-methyloctanoyl; Pos-6=D-Leu; Pos-7=Leu], polymyxin E₁-Ile [fatty acyl=(S)-6-methyloctanoyl; Pos-6=D-Leu; Pos-7=Ile], polymyxin E₁-Val [fatty acyl=(S)-6-methyloctanoyl; Pos-6=D-Leu; Pos-7=Val], polymyxin E₁-Nva [fatty acyl=(S)-6-methyloctanoyl; Pos-6=D-Leu; Pos-7=Nva], polymyxin E₂-Ile [fatty acyl=6-methylheptanoyl; Pos-6=D-Leu; Pos-7=Ile], polymyxin E₂-Val [fatty acyl=6-methylheptanoyl; Pos-6=D-Leu; Pos-7=Val] and polymyxin E₈-Ile [fatty acyl=7-methylnanoyl; Pos-6=D-Leu; Pos-7=Ile]. Refer to Table 1 of Velkov et al. (2013) ibid.

Polymyxin E/colistin acts on the bacterial cell membrane, interacting with lipopolysaccharide molecules in the outer membrane and resulting in increased cell permeability, leakage of cell-contents, lysis of cell, and finally, bacterial cell death (Velkov et al. (2013) ibid). Hydrophilic antibiotics (rifampicin, carbapenems, glycopeptides, and tetracyclines) can work synergistically owing to the disruption of membrane integrity by colistin. The use of colistin declined from 1970s to the early 2000s as safer/less-toxic aminoglycosides and anti-pseudomonal agents became available (Li et al. (2006) ibid).

Colistin is a multi-component polypeptide antibiotic composed of two cyclic peptides, colistin A and colistin B. Colistimethate sodium is the administered form of colistin, which is converted in vivo to form colistin. Colistin can be administered orally, topically (as otic solution and skin powder as colistin sulfate), intramuscularly, via inhalation, intrathecally, and intravenously as colistimethate sodium. Colistin is mostly active against Gram negative clinical isolates including Enterobacteriaceae. The non-fermentative P. aeruginosa and Acinetobacter species are naturally susceptible to colistin. Colistin is also effective against Haemophilus influenzae, E. coli, Salmonella spp., Shigella spp., Klebsiella spp., Legionella pneumophila, Aeromonas spp., Citrobacter spp., Bordetella pertussis, and Campylobacter species.

### Articles of Manufacture

The present invention further provides articles of manufacture comprising package material containing a salicylamide compound and an agent that increases the permeability of a bacterial cell membrane together with instructions for use in treating or preventing a bacterial infection in a patient or for reducing or eliminating formation of a bacterial biofilm, wherein the infection or biofilm comprises one or more Gram negative bacteria. Examples of salicylamide compounds (e.g. niclosamide, oxyclozanide, nitazoxanide, closantel) and examples of agents that increase the permeability of bacterial cell membranes (e.g. drug based agents including polymyxin B and polymyxin E) are given in further detail elsewhere.

The salicylamide compound and the agent that increases the permeability of a bacterial cell membrane may be administered separately, sequentially or simultaneously. For example, the combination of the salicylamide compound and the agent that increases the permeability of a bacterial cell membrane may be formulated together as a composition for administration to a patient. Alternatively, the salicylamide compound and the agent that increases the permeability of a bacterial cell membrane may each be separately formulated for separate or sequential administration to a patient.

The salicylamide compound and the agent that increases the permeability of a bacterial cell membrane may be administered to a patient by a variety of routes, including orally, parenterally, by inhalation spray, topically, rectally, nasally, buccally, intravenously, intra-muscularly, intra-dermally, subcutaneously or via an implanted reservoir, preferably intravenously. The amount of each compound to be administered will vary widely according to the nature of the patient and the nature and extent of the disorder to be treated. Typical dosages for an adult human will be 0.001 μg/mL to 100 μg/mL for the salicylamide compound and for the agent that increases the permeability of a bacterial cell membrane. The specific dosages required for any particular patient will depend upon a variety of factors, including the patient's age, body weight, general health, sex, etc.

For separate, sequential or simultaneous oral administration the salicylamide compound and the agent that increases the permeability of a bacterial cell membrane can be formulated into solid or liquid preparations, for example tablets, capsules, powders, solutions, suspensions and dispersions. Such preparations are well known in the art as are other oral dosage regimes not listed here. In the tablet form the compounds may be tableted with conventional tablet bases such as lactose, sucrose and corn starch, together with a binder, a disintegration agent and a lubricant. The binder may be, for example, corn starch or gelatin, the disintegrating agent may be potato starch or alginic acid, and the lubricant may be magnesium stearate. For oral administration in the form of capsules, diluents such as lactose and dried corn-starch may be employed. Other components such as colourings, sweeteners or flavourings may be added.

When aqueous suspensions are required for oral use, the salicylamide compound or the salicylamide compound and the agent that increases the permeability of a bacterial cell membrane may be combined with carriers such as water and ethanol, and emulsifying agents, suspending agents and/or surfactants may be used. Colourings, sweeteners or flavourings may also be added.

The salicylamide compound and the agent that increases the permeability of a bacterial cell membrane may also be administered separately, sequentially or simultaneously, by injection in a physiologically acceptable diluent such as water or saline. The diluent may comprise one or more other ingredients such as ethanol, propylene glycol, an oil or a pharmaceutically acceptable surfactant. In one example, the compounds are administered separately, sequentially or simultaneously by intravenous injection, where the diluent comprises an aqueous solution of sucrose, L-histidine and a pharmaceutically acceptable surfactant, e.g. Tween 20.

The salicylamide compound and the agent that increases the permeability of a bacterial cell membrane may also be administered, separately, sequentially or simultaneously, topically. Carriers for topical administration of the compounds include mineral oil, liquid petrolatum, white petrolatum, propylene glycol, polyoxyethylene, polyoxypropylene compound, emulsifying wax and water. The compounds may be present as ingredients in lotions or creams, for topical administration to skin or mucous membranes. Such creams may contain the active compounds suspended or dissolved in one or more pharmaceutically acceptable carriers. Suitable carriers include mineral oil, sorbitan monostearate, polysorbate 60, cetyl ester wax, cetearyl alcohol, 2-octyldodecanol, benzyl alcohol and water.

The salicylamide compound and the agent that increases the permeability of a bacterial cell membrane may further be administered separately, sequentially or simultaneously, by means of sustained release systems. For example, they may be incorporated into slowly dissolving tablets or capsules.

For the treatment of infections in plants, for example bacterial infections caused by Pseudomonas syringae pv. actinidiae (Psa-V) in kiwifruit plants of the genus Actinidia, the salicylamide compound and the agent that increases the permeability of a bacterial cell membrane may optionally be formulated with one or more carriers, for example as a spray for application to plants. The compounds may be applied separately, sequentially or simultaneously. For application to plants, the combinations and compositions of the invention may further comprise one or more adjuvants, such as emulsifiers, dispersants, mineral and vegetable oils, or mixtures thereof suitable for application to plants. The combinations and compositions can also be used as sterilising agents for field equipment (e.g. pruning shears), to prevent spreading of bacterial infections between orchards.

For the treatment of infections in animals, for example mastitis infections caused by gram negative bacteria, including E. coli, in dairy cows, the salicylamide compound and the agent that increases the permeability of a bacterial cell membrane may optionally be formulated with one or more carriers, for example as a spray for application to cow teats/udders. The compounds may be applied separately, sequentially or simultaneously, and may further comprise one or more adjuvants, such as emulsifiers, dispersants, mineral and vegetable oils, or mixtures thereof suitable for application to animals.

The present invention also relates to devices and kits for treating or preventing bacterial infections. Suitable kits comprise at least one salicylamide compound and at least one agent that increases the permeability of a bacterial cell membrane sufficient treatment of at least one bacterial infection, for separate, sequential or simultaneous use, together with instructions for performing the treatment/prevention.

The instructions for use of the kit and treating/preventing the bacterial infection can be in the form of labelling, which refers to any written or recorded material that is attached to, or otherwise accompanies a kit at any time during its manufacture, transport, sale or use. For example, the term “labelling” encompasses advertising leaflets and brochures, packaging materials, instructions, audio or video cassettes, computer discs, as well as writing imprinted directly on kits.

The Applicants' results presented herein provide surprisingly interesting insight into the molecular basis of how bacteria metabolise certain drugs, including nitro-prodrug antibiotics as well as salicylamides that contain one or more nitro groups, e.g. niclosamide and niclosamide analogues. Without wishing to be bound by theory, the applicants propose that bacteria that have become resistant to treatment with nitro-prodrug antibiotics by spontaneous mutations in endogenous nitroreductase genes would then be susceptible to treatment with one or more nitro group-containing salicylamide compounds owing to loss of ability to detoxify that compound via nitro-reduction. On the one hand, loss of, or reduction in, endogenous nitroreductase activity compared to wild type means that the bacterial cell is resistant to nitro-prodrug antibiotics, because once inside the bacterial cell the prodrug has no way of being cleaved to produce the toxic antibiotic (e.g., as described for a nitroreductase-deficient strain of E. coli with the 5-nitroimidazole antibiotic prodrug tinidazole by Prosser, G. A., Williams, E., M., Sissons, J., A., Walmsley, K., E., Parker, M., R., and Ackerley, D. F. (2015). A gain-of-function positive-selection expression plasmid that enables high-efficiency cloning. Biotechnology Letters 37:383-389). On the other hand, loss of, or reduction in, endogenous nitroreductase activity means that the bacterial cell is more susceptible to one or more nitro group-containing salicylamide compounds, for example niclosamide and niclosamide analogs, because in the absence of nitroreductase activity the bacterial cell is no longer capable of converting the toxic niclosamide to a non-toxic nitro-reduced form. A membrane permeabilizer may optionally be included with the one or more nitro group-containing salicylamide compounds to enhance sensitivity to the drug.

Accordingly, in yet another aspect the present invention provides a method for treating or preventing a bacterial infection in a patient, wherein the bacteria have become resistant to treatment with a nitro-prodrug antibiotic, comprising administering to the patient at least one salicylamide compound in an amount sufficient to treat or prevent infection, wherein the salicylamide compound includes one or more nitro group. Optionally, the method further comprises administering at least one membrane permeabilizer.

In yet another aspect the present invention provides a method for reducing or eliminating formation of a bacterial biofilm, wherein the bacteria have become resistant to treatment with a nitro-prodrug antibiotic, comprising administering at least one salicylamide compound in an amount sufficient to reduce or eliminate formation of the biofilm, salicylamide compound includes one or more nitro group. Optionally, the method further comprises administering at least one membrane permeabilizer.

Conversely, bacteria that have become resistant to treatment with one or more nitro group-containing salicylamide compounds may have done so via mutations in endogenous nitroreductase genes that cause an increase in nitroreductase enzyme activity. On the one hand, an increase in endogenous nitroreductase activity compared to wild type means that the bacterial cell is resistant to nitro group-containing salicylamide compounds because the bacterial cell is no longer capable of converting the toxic nitro group-containing salicylamide compound, for example niclosamide and niclosamide analogs, to a non-toxic form. On the other hand, an increase in endogenous nitroreductase activity means that the bacterial cell is more susceptible to one or more nitro-prodrug antibiotics, because it will activate the prodrug to form an active form of the antibiotic.

Accordingly, in yet another aspect the present invention provides a method for treating or preventing a bacterial infection in a patient, wherein the bacteria have become resistant to treatment with at least one salicylamide compound and at least one agent that increases the permeability of a bacterial cell membrane, wherein the salicylamide compound includes one or more nitro groups, comprising administering to the patient a nitro-prodrug antibiotic in an amount sufficient to treat or prevent the infection.

In yet another aspect the present invention provides a method for reducing or eliminating formation of a bacterial biofilm, wherein the bacteria have become resistant to treatment with at least one salicylamide compound or the combination of at least one salicylamide compound and at least one agent that increases the permeability of a bacterial cell membrane, wherein the salicylamide compound includes one or more nitro groups, comprising administering a nitro-prodrug antibiotic in an amount sufficient to reduce or eliminate formation of the biofilm.

### Articles of Manufacture: Medical Devices

The compositions and combinations according to the present invention may be disposed on indwelling medical devices and the like to prevent or treat infection caused by Gram negative bacteria. This includes formation of bacterial biofilms, for example, post-surgery or operative procedure. In certain aspects, the compositions and combinations according to the present invention may be disposed on catheters, stents, medical implants (e.g. artificial hips and the like) in order to guard against infection.

A person skilled in the art will understand that that compositions and combinations according to the present invention may be formulated in such a way that optimizes their antibacterial activity, (e.g.) as a coating on a medical device such as an implant, stent, medical implant etc.

### Dosage Forms and Formulations and Administration

The compounds of the invention may be present in an isolated or substantially or essentially pure form. It will be understood that the product may be mixed with carriers or diluents that will not interfere with the intended purpose of the product and still be regarded as isolated or substantially pure. A product of the invention may also be in a substantially or essentially purified form, preferably comprising or consisting essentially of about 80%, 85%, or 90%, e.g. at least about 95%, at least about 98% or at least about 99% of the compound or dry mass of the preparation.

Depending on the intended route of administration, the pharmaceutical products, pharmaceutical compositions, combined preparations and medicaments of the invention may, for example, take the form of solutions, suspensions, installations, sustained release formulations, or powders, and typically contain about 0.1%-95% of active ingredient(s), preferably about 0.2%-70%. Other suitable formulations include injection- and infusion-based formulations. Other useful formulations include sustained release preparations, including, for example, controlled, slow or delayed release preparations.

Aspects of the invention include controlled or other doses, dosage forms, formulations, compositions and/or devices containing a salicylamide compound and an agent that increase the permeability of a bacterial cell membrane. The present invention includes, for example, doses and dosage forms for at least oral administration, transdermal delivery, topical application, suppository delivery, transmucosal delivery, injection (including subcutaneous administration, subdermal administration, intramuscular administration, depot administration, and intravenous administration, including delivery via bolus, slow intravenous injection, and intravenous drip), infusion devices (including implantable infusion devices, both active and passive), administration by inhalation or insufflation, buccal administration and sublingual administration. It will be appreciated that any of the dosage forms, compositions, formulations or devices described herein particularly for intravenous administration may be utilized, where applicable or desirable, in a dosage form, composition, formulation or device for administration by any of the other routes herein contemplated or commonly employed. For example, a dose or doses could be given parenterally using a dosage form suitable for parenteral administration which may incorporate features or compositions described in respect of dosage forms suitable for oral administration, or be delivered in an sustained dosage form, such as a modified release, extended release, delayed release, slow release or repeat action dosage form.

Preferably the salicylamide compound and an agent that increase the permeability of a bacterial cell membrane of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or pH buffering agents may also be present.

The term “pharmaceutically acceptable carrier” refers to any useful carriers, excipients, or stabilizers which are non-toxic to the host cell or non/human animal being exposed thereto at the dosages and concentrations employed, and include pharmaceutical carriers that do not induce the production of antibodies harmful to the individual receiving the composition. Suitable carriers can be large, slowly metabolized macromolecules such as proteins, polysaccharides, polylactic acids, polyglycolic acids, polymeric amino acids, and amino acid copolymers. Often the physiologically acceptable carrier is an aqueous pH buffered solution. Other examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, polyethylene glycol (PEG), and Pluronics.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

An auxiliary agent such as casein, gelatin, albumin, glue, sodium alginate, carboxymethylcellulose, methylcellulose, hydroxyethylcellulose or polyvinyl alcohol may also be included in the formulation of the invention.

The dosage forms, formulations, devices and/or compositions of the invention may be formulated to optimize bioavailability and to maintain plasma concentrations within the therapeutic range, including for extended periods. Sustained delivery preparations, e.g., controlled delivery preparations, also optimize the drug concentration at the site of action and minimize periods of under and over medication, for example.

The dosage forms, devices and/or compositions useful in the invention may be provided for periodic administration, including once daily administration, for low dose controlled and/or low dose long-lasting in vivo release of the salicylamide compound and agent that increases the permeability of a bacterial cell membrane.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of the salicylamide compound and agent that increase the permeability of a bacterial cell membrane.

Examples of dosage forms suitable for transdermal administration include, but are not limited to, transdermal patches, transdermal bandages, and the like. Examples of dosage forms suitable for topical administration of the compounds and formulations useful in the invention are any lotion, stick, spray, ointment, paste, cream, gel, etc., whether applied directly to the skin or via an intermed.

Examples of dosage forms suitable for suppository administration of the compounds and formulations useful in the invention include any solid dosage form inserted into a bodily orifice particularly those inserted rectally, vaginally and urethrally.

Examples of dosage forms suitable for transmucosal delivery of the compounds and formulations useful in the invention include depositories solutions for enemas, pessaries, tampons, creams, gels, pastes, foams, nebulised solutions, powders and similar formulations containing in addition to the active ingredients such carriers as are known in the art to be appropriate.

Examples of dosage of forms suitable for injection of the compounds and formulations useful in the invention include delivery via bolus such as single or multiple administrations by intravenous injection, subcutaneous, subdermal, and intramuscular administration or oral administration.

Examples of dosage forms suitable for depot administration of the compounds and formulations useful in the invention include pellets or small cylinders of active agent or solid forms wherein the active agent is entrapped in a matrix of biodegradable polymers, microemulsions, liposomes or is microencapsulated.

Examples of infusion devices for compounds and formulations useful in the invention include infusion pumps containing the salicylamide compound and agent that increase the permeability of a bacterial cell membrane and/or pre-complexed compounds/agents, at a desired amount for a desired number of doses or steady state administration, and include implantable drug pumps.

Examples of implantable infusion devices for compounds and formulations useful in the invention include any solid form in which the active agent is encapsulated within or dispersed throughout a biodegradable polymer or synthetic, polymer such as silicone, silicone rubber, silastic or similar polymer.

Examples of dosage forms suitable for inhalation or insufflation of compounds and formulations useful in the invention include compositions comprising solutions and/or suspensions in pharmaceutically acceptable, aqueous, or organic solvents, or mixture thereof and/or powders.

Examples of dosage forms suitable for buccal administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of dosage forms suitable for sublingual administration of the compounds and formulations useful in the invention include lozenges, tablets and the like, compositions comprising solutions and/or suspensions in pharmaceutically acceptable, aqueous, or organic solvents, or mixtures thereof and/or powders.

Examples of controlled drug formulations for delivery of the compounds and formulations useful in the invention are found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C., Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. Further guidance concerning the analysis of extended release dosage forms has been provided by the FDA. See Guidance for Industry. Extended release oral dosage forms: development, evaluation, and application of in vitro/in vivo correlations. Rockville, Md.: Center for Drug Evaluation and Research, Food and Drug Administration (1997).

Further examples of dosage forms useful in the methods of the invention include, but are not limited to, modified-release (MR) dosage forms including delayed-release (DR) forms; prolonged-action (PA) forms; controlled-release (CR) forms; extended-release (ER) forms; timed-release (TR) forms; and long-acting (LA) forms. For the most part, these terms are used to describe orally administered dosage forms, however these terms may be applicable to any of the dosage forms, formulations, compositions and/or devices described herein. These formulations effect delayed total drug release for some time after drug administration, and/or drug release in small aliquots intermittently after administration, and/or drug release slowly at a controlled rate governed by the delivery system, and/or drug release at a constant rate that does not vary, and/or drug release for a significantly longer period than usual formulations.

Modified-release dosage forms of the invention include dosage forms having drug release features based on time, course, and/or location which are designed to accomplish therapeutic or convenience objectives not offered by conventional or immediate-release forms. See, for example, Bogner, R. H. U.S. Pharmacist 22 (Suppl.):3-12 (1997); Scale-up of oral extended-release drug delivery systems: part I, an overview, Pharmaceutical Manufacturing 2:23-27 (1985). Extended-release dosage forms of the invention include, for example, as defined by The United States Food and Drug Administration (FDA), a dosage form that allows a reduction in dosing frequency to that presented by a conventional dosage form, e.g., a solution or an immediate-release dosage form. See, for example, Bogner, R. H. (1997) supra. Repeat action dosage forms of the invention include, for example, forms that contain two single doses of medication, one for immediate release and the second for delayed release. Bi-layered tablets, for example, may be prepared with one layer of drug for immediate release with the second layer designed to release drug later as either a second dose or in an extended-release manner. Targeted-release dosage forms of the invention include, for example, formulations that facilitate drug release and which are directed towards isolating or concentrating a drug in a body region, tissue, or site for absorption or for drug action.

Also useful in the invention are coated beads, granules or microspheres containing a salicylamide compound and agent that increases the permeability of a bacterial cell membrane and/or pre-complexed compound/agent, which may be used to achieve modified release by incorporation of the drug into coated beads, granules, or microspheres. In such systems, the salicylamide compound and agent that increases the permeability of a bacterial cell membrane is distributed onto beads, pellets, granules or other particulate systems. See Ansel, H. C., Allen, L. V. and Popovich, N. G., Pharmaceutical Dosage Forms and Drug Delivery Systems, 7th Ed., Lippincott 1999, p. 232.

A number of methods may be employed to generate modified-release dosage forms of the salicylamide compound and agent that increases the permeability of a bacterial cell membrane suitable for oral administration to humans and other animals. Two basic mechanisms available to achieve modified release drug delivery include altered dissolution or diffusion of drugs and excipients. Within this context, for example, four processes may be employed, either simultaneously or consecutively. These are as follows: (i) hydration of the device (e.g., swelling of the matrix); (ii) diffusion of water into the device; (iii) controlled or delayed dissolution of the drug; and (iv) controlled or delayed diffusion of dissolved or solubilized drug out of the device.

In order to formulate a range of dosage values, cell culture assays and animal studies can be used. The dosage of such compounds preferably lies within the dose that is therapeutically effective for at least 50% of the population, and that exhibits little or no toxicity at this level.

The effective dosage of the salicylamide compound and agent that increases the permeability of a bacterial cell membrane employed in the methods and compositions of the invention may vary depending on a number of factors including the particular the salicylamide compound and agent that increases the permeability of a bacterial cell membrane employed, the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient.

A suitable dose may be from about 0.001 to about 1 or to about 10 mg/kg body weight such as about 0.01 to about 0.5 mg/kg body weight. A suitable dose may however be from about 0.001 to about 0.1 mg/kg body weight such as about 0.01 to about 0.05 mg/kg body weight. Doses from about 1 to 100, 100-200, 200-300, 300-400, and 400-500 miligrams are appropriate, as are doses of about 500-750 micrograms and about 750-1000 micrograms. Other useful doses include from about 300 to about 1000 picomoles per dose, and about 0.05 to about 0.2 nanomoles per dose. Still other doses are within the following claims.

Doses may be administered in single or divided applications. The doses may be administered once, or application may be repeated.

The routes of administration and dosages described herein are intended only as a guide since a skilled physician will consider the optimum route of administration and dosage for any particular patient and condition.

Any reference to prior art documents in this specification is not to be considered an admission that such prior art is widely known or forms part of the common general knowledge in the field.

The invention is further described with reference to the following Examples. It will be appreciated that the invention as claimed is not intended to be limited in any way by these Examples.

### EXAMPLES

### Example 1: Antibiotic Combinations/Growth Assays

This procedure applies to the experiments depicted in FIGS. 1-52. The desired bacterial strain is inoculated into 3 mL LB and incubated for 16 hours at 30° C. with shaking at 250 rpm. 500 μL aliquots of each overnight culture are used to inoculate 20 ml of LB in 50 mL bioreactor tubes and incubated at 30° C., 250 rpm for 2 h or as otherwise stated in the Figure Description. 40 μL of each culture is then added to a multiplex assay (in quadruplicate) of salicylamide (e.g. niclosamide, oxyclozanide, nitazoxanide or closantel) and an agent that increases the permeability of a bacterial cell membrane (e.g., polymyxin B, polymyxin E (colistin), gramicidin) in a 384 well plate format. Each well of the 384 well plate contains 40 μL LB amended with double the final desired concentration of the desired drug combination to allow for a 1 in 2 dilution with bacterial culture. The OD600 is measured (t=0) and the cultures are incubated at 30° C., 1200 rpm for 4 hours or as otherwise stated in the Figure Description. The final OD600 (at e.g. t=4) is then recorded. To calculate growth inhibition the t=0 value is subtracted from the final OD600 value. The percentage growth is then calculated relative to the 0 μM drug combination well, which represents 100% growth.

The testing format is a two dimensional 384 well plate assay where replica cultures of each test strain are challenged with increasing concentrations of membrane permeabilizer on the horizontal axis, and increasing concentrations of salicylamide on the vertical axis (each prepared as a two-fold dilution series, from right-to-left for membrane permeabilizers and bottom-to-top for the salicylamide).

The results are presented in FIGS. 1-56.

Specifically, niclosamide when used in conjunction with a polymyxin, such as (e.g.) polymyxin B or polymyxin E (colistin) provides a synergistic growth inhibition effect on a range of different Gram negative bacteria including, but not limited to, both antibiotic-resistant and laboratory strains of Escherichia coli, both antibiotic-resistant and laboratory strains of Pseudomonas aeruginosa, both antibiotic-resistant and laboratory strains of Klebsiella pneumoniae, Enterobacter cloacae, Salmonella enterica, and Acinetobacter baumannii (FIGS. 1-40).

The generic ability of membrane permeabilizing drugs beyond the polymyxin family to sensitize Gram negative bacteria to the antibiotic effects of salicylamide drugs was further demonstrated using the membrane-permeabilizing antibiotic gramicidin (a mixture of gramicidin A, B, C and D; catalog# G5002 from Sigma-Aldrich). Gramicidin provides a synergistic growth inhibition effect on a range of different Gram negative bacteria including, but not limited to, E. coli laboratory strain W3110 and an antibiotic-resistant clinical isolate of Pseudomonas aeruginosa (FIGS. 41-44).

Further, the effect of oxyclozanide (FIGS. 45-52), nitazoxanide (FIGS. 53 and 54), and closantel (FIGS. 55 and 56) demonstrate that salicylamide compounds related to niclosamide exhibit similar synergistic effects when used in combination with various membrane permeabilizing antibiotics (e.g. colistin) against a wide range of Gram negative bacteria.

FIGS. 57 and 58 demonstrate that the combination of colistin and a membrane-uncoupling agent, namely, 2,4-dinitrophenol, exhibited only weak synergistic effect and does not achieve the same level of growth inhibition against E. coli, at similar concentration when compared to the combination of colistin and niclosamide (e.g. FIGS. 1, 2, 5, 6, 9, 10), or colistin and oxyclozanide (FIGS. 45, 46), or colistin and nitazoxanide (FIGS. 53, 54), or colistin and closantel (FIGS. 55, 56).

### Example 2: In Vivo Real-Time Measurement of Intrabacterial Redox Potential

Generation of E. coli Redox Screening Strains

Redox screening strains were generated by in-frame deletion using PCR-amplified disruption cassettes through the Red recombinase method (Datsenko & Warner (2000) Proc Natl Acad Sci USA 97, 6640-6645). E. coli strain 7KO was derived from E. coli W3110 by deletion of the native nfsA, nfsB, azoR, nemA, yieF, ycaK and mdaB genes as previously described by Copp et al. (Copp et al. (2014) Protein Eng. Des. Sel. 27, 399-403). E. coli strain 7KOΔtolC was derived from 7KO by deletion of the native tolC gene. The pFPX25-roGFP2 vector (van der Heijden et al. (2015) Proc Natl Acad Sci USA 112, 560-565) was cloned into E. coli strains 7KO and 7KOΔtolC to generate 7KO:roGFP and 7KOΔtolC:roGFP respectively.

### In Vivo Real-Time Measurement of Intrabacterial Redox Potential

In vivo analysis of the intrabacterial redox potential was performed at 30° C. in a Synergy H1 Multimode plate reader (BioTek) with excitation measured at 405 and 480 nm and emission measured at 510 nm. Log phase bacterial cultures of 7KO:roGFP or 7KOΔtolC:roGFP were resuspended in 0.9% saline at OD 1.0, and 100 μL aliquots were loaded in individual wells of a black, clear-bottom 96-well plate. Background signals from the non-fluorescent (i.e., without pRSETB-RoGFP2) corresponding strain were obtained in the same experiment. Fluorescence signals were followed for 160-180 min, and the resulting 405/480 ratio signals were calculated. At 10 min, duplicate aliquots were challenged with niclosamide, and/or an agent that increases the permeability of a bacterial cell membrane (e.g., polymyxin B, or polymyxin E (colistin)), and/or an agent that inhibits the TolC efflux pump of Gram negative bacteria (e.g., PABN) in a 96 well plate format. Reduced and oxidized controls were obtained within each experiment using a final concentration of 10 mM H₂O₂ or 1 mM DTT respectively. All values were normalized to the values obtained for maximally oxidized and for fully reduced bacterial cultures.

The results are presented in FIGS. 59 and 60.

Specifically, niclosamide causes an irreversible increase in intracellular redox stress when administered in combination with a membrane permeabilizing agent (e.g., polymyxin B, or polymyxin E (colistin)), or an agent that inhibits the TolC efflux pump of Gram negative bacteria (e.g., PAβN), or when administered in a strain with impaired efflux mechanisms (e.g., E. coli strain 7KOΔtolC, which harbors an in-frame deletion of the native tolC gene).

### AMNIS ImageStream and IDEAS/ImageJ Analysis

Samples were analyzed by the AMNIS ImageStream as previously described (van der Heijden et al. (2015) Proc Natl Acad Sci USA 112, 560-565). The laser intensities for 405, 488, 658, and 785 nm were 100, 120, 20, and 3.8, respectively. The data files were further analyzed using IDEAS software, version 6.0.129.0, which is supplied by AMNIS. Bacterial cells were selected based on fluorescence at 660 nm. Every image of an infected cell was then selected by the program based on fluorescent intensity at 660 nm. Based on this selection, a mask was created that was used for analysis of the 405/480 nm ratio. The resulting 405/480 ratio signals were plotted in a histogram. Reduced and oxidized controls were obtained within each experiment, and all values were normalized to oxidized and reduced ratio values. Pseudocolored ratio images were made through analysis using ImageJ as was described previously (Morgan et al. (2011) Free Radical Biology and Medicine 51, 1943-1951).

In vivo analysis of the intrabacterial redox potential was done at 30° C. in a Synergy H1 Multimode plate reader (BioTek) with excitation at 405 and 480 nm while emission was measured at 510 nm. Log phase bacterial cultures were resuspended in 0.9% saline at OD 1.0, and 200 μL per well were loaded in a black, clear-bottom 96-well plate. Background signals from the nonfluorescent corresponding strain were obtained in the same experiment. Additionally, the signals for fully oxidized and fully reduced bacteria were obtained by adding 10 mM H₂O₂ and 1 mM DTT respectively to the bacterial culture at the start of the experiment. All values were normalized to the values obtained for maximally oxidized and for fully reduced bacterial cultures.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A pharmaceutical composition comprising a salicylamide compound and an agent that increases the permeability of a bacterial cell membrane, provided that the pharmaceutical composition does not contain niclosamide and colistin.

2. A pharmaceutical composition according to claim 1, wherein the salicylamide compound is selected from the group consisting of niclosamide, oxyclozanide, nitazoxanide and closantel.

3. A pharmaceutical composition according to claim 2, wherein the agent that increases the permeability of a bacterial cell membrane is a polymyxin, or cationic or anionic peptides that disrupt cell membrane homeostasis and/or polarity including gramicidin.

4. A pharmaceutical composition according to claim 3, wherein the polymyxin is polymyxin B or polymyxin E, including colistin.

5. A pharmaceutical composition according to any one of claims 1 to 4, further comprising a pharmaceutically acceptable excipient.

6. A combination product, a synergistic combination, an anti-bacterial agent or a composition comprising a salicylamide compound and an agent that increases the permeability of a bacterial cell membrane, provided that the combination product, a synergistic combination, an anti-bacterial agent or a composition does not contain niclosamide and colistin.

7. A pharmaceutical composition according to any one of claims 1 to 5 or a combination product, a synergistic combination, an anti-bacterial agent or a composition according to claim 6 for use in:
(i) treating or preventing a bacterial infection in a human or non-human animal; or
(ii) reducing or eliminating formation of a bacterial biofilm
wherein the infection or biofilm comprises one or more Gram negative bacteria.

8. A method for treating or preventing a bacterial infection in a patient or for reducing or eliminating formation of a bacterial biofilm comprising administering an antibiotically effective amount of a salicylamide compound and an agent that increases the permeability of a bacterial cell membrane, wherein the infection or biofilm comprises one or more Gram negative bacteria.

9. A method according to claim 8, wherein the salicylamide compound is selected from the group consisting of niclosamide, oxyclozanide, nitazoxanide and closantel.

10. A method according to claim 8 or claim 9, wherein the agent that increases the permeability of the bacterial cell membrane is a polymyxin, or cationic or anionic peptides that disrupt cell membrane homeostasis and/or polarity including gramicidin.

11. A method according to claim 10, wherein the polymyxin is polymyxin B or polymyxin E.

12. A method according to any one of claims 8 to 11, wherein the Gram negative bacterium is selected from the group consisting of Escherichia coli, Enterobacter cloacae, Salmonella enterica, Pseudomonas aeruginosa, Pseudomonas syringae, Klebsiella pneumonia, Acinetobacter baumannii.

13. A method according to any one of claims 8 to 12, wherein the salicylamide compound and the agent that increases the permeability of a bacterial cell membrane are used in proportions sufficient to produce a synergistic antibiotic effect.

14. A method according to any one of claims 8 to 13, wherein the salicylamide compound and the agent that increases the permeability of a bacterial cell membrane are administered to a patient via a route selected from oral, parenteral, by inhalation spray, topical, rectal, nasal, buccal, intravenous, intra-muscular, intra-dermal, subcutaneous or via an implanted reservoir.

15. An article of manufacture comprising package material containing a salicylamide compound and an agent that increases the permeability of a bacterial cell membrane, together with instructions for use in treating or preventing a bacterial infection in a patient or for reducing or eliminating formation of a bacterial biofilm, wherein the infection or biofilm comprises one or more Gram negative bacteria, provided that the package material does not contain niclosamide and colistin.
